(19) United States
(12) Patent Application Publication
(10) Pub. No.: US 2015/0045389 A1
(43) Pub. Date: Feb. 12, 2015

(54) COMPOUNDS AND METHODS FOR INHIBITING CIF VIRULENCE FACTOR

(71) Applicants: Trustees of Dartmouth College, Hanover, NH (US); The Regents of the University of California, Oakland, CA (US)

(72) Inventors: Dean R. Madden, Hanover, NH (US); Christopher D. Bahl, Enfield, NH (US); Bruce D. Hammock, Davis, CA (US); Christophe Morisseau, West Sacramento, CA (US)

(21) Appl. No.: 14/386,850

(22) PCT Filed: Apr. 9, 2013
(86) PCT No.: PCT/US13/35735

§ 371 Date: Sep. 22, 2014

Related U.S. Application Data
(60) Provisional application No. 61/622,198, filed on Apr. 10, 2012.
(60) Provisional application No. 61/711,394, filed on Oct. 9, 2012.

(51) Int. Cl. C07D 231/12; C07D 211/96; C12Q 1/34; C07C 59/135; C07C 275/28; C07C 275/30; C07D 211/58; C07C 229/08

(52) U.S. Cl. CPC C07D 231/12; C07D 211/58; C07D 211/96; C07C 229/08; C07C 59/135; C07C 275/28; C07C 275/30; C12Q 1/34; G01N 2500/02; G01N 2800/382
USPC 514/319; 514/329; 514/406; 514/567; 514/570; 514/596; 435/18; 435/184; 562/499; 546/224; 548/377.1; 562/447; 562/478; 564/55; 564/53

(57) ABSTRACT

The present invention is a screening assay for identifying inhibitors of Pseudomonas aeruginosa CFTR Inhibitory Factor as well as compounds identified by the screening assay for use in compositions and methods for ameliorating or treating a respiratory disease such as cystic fibrosis or secondary infection thereof.

### INTRODUCTION

This invention was made with government support under contract numbers T32-AI007519, T32-DK007301, R01-AI091699, R01-DK075309 and R01-ES002710 awarded by the National Institutes of Health. The government has certain rights in the invention. Work on this invention was also supported by grants from the Cystic Fibrosis Foundation.

### BACKGROUND OF THE INVENTION

Pseudomonas aeruginosa is a Gram-negative opportunistic pathogen that commonly causes ocular and pulmonary infections, as well as burn wound infections. This bacterium possesses an inherent resistance to most antibiotics, and is able to form biofilms that further enhance antibiotic resistance and chronic infections (Davies & Bilton (2009) Respir. Care 54:628-40). Of particular clinical importance is the prominence of P. aeruginosa infection in patients with compromised pulmonary function. By the age of 18, 80% of all patients with cystic fibrosis (CF) have a chronic P. aeruginosa lung infection (Geller (2009) Respir. Care 54:658-70). Furthermore, chronic obstructive pulmonary disorder (COPD) is the fourth leading cause of death world-wide (Vogt, et al. (2009) S D Med. Spec No 30-7), and P. aeruginosa pulmonary infection in these patients results in a rapid decline in lung function and a poor long-term prognosis (Murphy, et al. (2008) Am. J. Respir. Crit. Care Med. 177:853-60). P. aeruginosa also causes many nosocomial infections, exacerbating ventilator-associated pneumonias and hospital-acquired pneumonia (Zavascki, et al. (2006) Crit. Care 10:R114). Recently, a more aggressive strain of P. aeruginosa emerged in Liverpool, England that caused an epidemic in the CF community (Salunkhe, et al. (2005) J. Bacteriol. 187:4908-20). Therefore, finding new and effective ways to prevent and treat P. aeruginosa infection will help to reduce human morbidity and mortality.

During the course of infection, P. aeruginosa produces and secretes an arsenal of toxins and virulence factors (Kipnis, et al. (2006) Med. Mal. Infect. 36:78-91; Bleves, et al. (2010) Int. J. Med. Microbiol. 300:534-43). Of particular interest is the virulence factor Cif (Cystic fibrosis transmembrane conductance regulator Inhibitory Factor), an epoxide hydrolase (EH) that enters human cells and prevents the deubiquitination of the cystic fibrosis transmembrane conductance regulator (CFTR) (Bomberger, et al. (2011) PLoS Pathog 7:e1001325). Patients with CF have a mutation in the chloride ion channel CFTR that prevents its function and/or localization to the apical surface of airway epithelial cells, resulting in an osmotic imbalance that dehydrates the airway surface liquid and prevents mucociliary clearance (Rogan, et al. (2011) Chest 139:1480-90). Cif induces a rapid decline in cell surface CFTR levels (MacEachran, et al. (2007) Infect. Immun. 75:3902-12), essentially phenocopying the genetic disorder CF (Swiatecka-Urban, et al. (2006) Am. J. Physiol. Cell Physiol. 290:C862-72). In patients with wild-type CFTR, Cif maintains a persistent infection. In patients with CF that have a P. aeruginosa infection, Cif could greatly impede the efficacy of therapies designed to rescue CFTR function. Cif has also been shown to affect other ABC transporters (Ye, et al. (2008) Am. J. Physiol. Cell Physiol. 295:C807-818), suggesting that it may have additional deleterious effects on cellular physiology in vivo.

Cif is the first reported example of an epoxide hydrolase utilized as a bacterial virulence factor (Bahl, et al. (2010) J. Bacteriol. 192:1785-95). Cif possesses the hallmark catalytic triad that is characteristic of α/β hydrolases, which includes a nucleophile and a charge relay His and acid (Holmquist (2000) Curr. Protein Pept. Sci. 1:209-235). Prior to structural elucidation, Cif's catalytic triad His was predicted to be at position 269 by sequence alignment, and this residue was mutated to Ala (MacEachran, et al. (2007) supra). Cif-H269A was found lacking in enzyme activity using the colorigenic EH substrate S-NEPC. This mutant protein was also shown to be deficient in lowering apical surface CFTR abundance of human cells, suggesting a link between EH enzyme activity and the cellular effects of Cif. However, when the structure of Cif was determined by X-ray crystallography, it became clear that His297 was in fact the catalytic triad His (Bahl, et al. (2010) J. Bacteriol. 192:1785-1795). The catalytic triad of Cif is buried within the core of the protein at the interface between the cap and core domains. However, His269 is located on the protein surface, and appears to be positioned at the mouth of the tunnel leading to the active site.

To analyze the Cif effect, an understanding of how Cif functions as an EH is needed. EHs are an extensively studied class of enzymes (Arand, et al. (2005) Methods Enzymol. 400:569-588; Arand, et al. (2003) Drug Metab. Rev. 35:365-383; Morisseau & Hammock (2005) Annu. Rev. Pharmacol. Toxicol. 45:311-333). The active site is sequestered within the interior of the protein, at the interface between the α/β hydrolase core domain and a cap domain. According to the canonical mechanism, an epoxide substrate enters the active site and is bound by a ring-opening pair of polar residues. A nucleophile attacks an epoxide carbon, opening the ring and forming a covalent intermediate (Pinot, et al. (1995) J. Biol. Chem. 270:968-7974). Further, a charge-relay His-acid pair activates a water molecule to nucleophilically attack the enzyme-substrate intermediate and release the hydrolysis product. While Cif exhibits multiple sequence and structural deviations from the archetypal EH active site (Bahl & Madden (2012) Protein Pept. Lett. 19:186-193; Bahl, et al. (2010) supra), their comparison nonetheless allows for a focused analysis.

### SUMMARY OF THE INVENTION

The present invention is a pharmaceutical composition formulated for pulmonary administration, which includes an inhibitor of Cif activity in admixture with a pharmaceutically acceptable carrier, wherein the inhibitor is a long chain or very long chain fatty acid monoepoxide or is a compound having the structure of Formula I

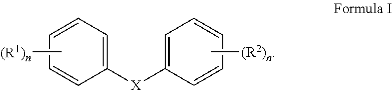

In certain embodiments, the inhibitor is tiratricol. In other embodiments, the pharmaceutical composition is used in a method for ameliorating or treating a respiratory disease (e.g., chronic obstructive pulmonary disease, pneumonia, an Acinetobacter infection, a P. aeruginosa infection or cystic fibrosis), or a secondary infection thereof (e.g., a viral infection, an Acinetobacter or P. aeruginosa infection).

Methods for inhibiting the activity of Cif and for identifying an inhibitor of Cif activity are also provided. In accordance with the instant screening assay, a prokaryotic Cif protein is contacted with a test compound in the presence of cyano(6-methoxynaphthalen-2-yl)methyl (oxiran-2-ylmethyl) (CMNGC); and it is determined whether the test compound inhibits hydrolysis of CMNGC.

### BRIEF DESCRIPTION OF THE DRAWINGS

FIG. 1 shows the inhibitory activity of fatty acid monoepoxides.

### DETAILED DESCRIPTION OF THE INVENTION

P. aeruginosa Cif, and Cif proteins from other bacteria, e.g., Acinetobacter sp. 13TU RUH2624, are epoxide hydrolases. It has now been found that these bacterial enzymes can be inhibited by tiratricol and a long chain or very long chain fatty acid monoepoxide. In addition to decreasing apical CFTR expression and reducing CFTR-mediated Cl⁻ ion secretion, Cif is able to, trigger the degradation of the transporter associated with antigen presentation 1 (TAP-1), which like CFTR is a member of the ABC transporter family. Because of its effect on TAP-1, Cif can impede the immune response to viral infections, which are major contributors to clinical exacerbations in patients with cystic fibrosis. Thus, an inhibitor of Cif enzyme activity is of use in reducing the ability of P. aeruginosa to infect patient airways; reducing the ability of P. aeruginosa to reverse the effects of CFTR corrector and potentiator compounds in patients with cystic fibrosis (CF); reducing the ability of P. aeruginosa to shield viral infections from immune surveillance and reducing infection by a bacterium that expresses a Cif enzyme.

A novel approach was employed to identify Cif inhibitors (iCifs). The assay utilized the fluorogenic reporter compound, cyano(6-methoxynaphthalen-2-yl)methyl (oxiran-2-ylmethyl), which binds the unusual active-site geometry of Cif. Using this reporter compound, together with a weak inhibitor as a positive control, the assay proved robust, and identified a compound, 3,3′,5-triiodothyroacetic acid (tiratricol), that potently (4 μM) inhibited Cif enzyme activity. Tiratricol was co-crystallized with the Cif protein, and shown to block the entrance of the tunnel leading to the active site of the enzyme.

Accordingly, the present invention pertains to a screening assay for identifying Cif inhibitors as well as compounds identified by the screening assay for use in compositions and methods for ameliorating or treating a respiratory disease or secondary infection thereof. In accordance with the instant screening assay, Cif protein is contacted with a test compound in the presence of CMNGC and it is determined whether the test compound inhibits hydrolysis of CMNGC, wherein a compound that inhibits hydrolysis of CMNGC is indicative of an inhibitor of Cif activity.

The prokaryotic Cif protein of use in the instant assay can be isolated from its natural source (i.e., P. aeruginosa or Acinetobacter) or can be produced by recombinant DNA methods or by synthetic chemical methods routinely practiced in the art. For recombinant production, prokaryotic Cif (e.g., P. aeruginosa, Accession No. Q9HZR3; Acinetobacter sp. 13TU RUH2624, Accession No. DOBWK6) can be expressed in known prokaryotic or eukaryotic expression construct systems. Bacterial, yeast, insect, or mammalian expression systems can be used, as is known in the art.

Alternatively, synthetic chemical methods, such as solid-phase peptide synthesis, can be used to synthesize Cif. General means for the production of proteins are outlined in B. Weinstein, ed., Chemistry and Biochemistry of Amino Acids, Peptides, and Proteins, a Survey of Recent Developments (1983).

Cif can be produced alone or as a fusion protein. A Cif fusion protein includes two protein segments, i.e., Cif fused to another protein segment by means of a peptide bond. The first protein segment includes a full-length Cif protein and can be on the N-terminus or C-terminus of the fusion proteins, as is convenient. The second protein segment can be a full-length protein or a protein fragment or polypeptide. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Epitope tags can be used in fusion protein constructions, including histidine (His) tags, FLAG tags (Kodak), influenza hemagglutinin (HA) tags, Myc tags, VSV-G tags, and thioredoxin (Trx) tags. Other fusion constructions can include maltose binding protein (MBP), S-tag, Lex A DNA binding domain (DBD) fusions, GAL4 DNA binding domain fusions, and herpes simplex virus (HSV) BP16 protein fusions.

Fusion proteins can be made by covalently linking the first and second protein segments or by standard procedures in the art of recombinant DNA technology. Recombinant DNA methods can be used to prepare fusion proteins, for example, by making a DNA construct which includes a nucleic acid sequence encoding Cif in proper reading frame with nucleotides encoding the second protein segment and expressing the DNA construct in a host cell, as is routinely practiced in the art. Many kits for constructing fusion proteins and expressing recombinant proteins are commercially available from companies such as Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada).

Expression of Cif or a Cif fusion protein can be carried out in any suitable host cell including prokaryotic or eukaryotic host cells. A variety of host cells for use in mammalian, yeast, bacterial, or insect expression systems are available and can be used to express the Cif or Cif fusion protein. Suitable mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, baby hamster kidney cells, HL-60, U937, HaK, or Jurkat cells.

Yeast or prokaryotic host cells can also be used. Suitable yeast strains include Saccharomyces cerevisiae, Schizosaccharomyces pombe, Kluyveromyces strains, Candida, or any yeast strain capable of expressing heterologous proteins. Suitable bacterial strains include Escherichia coli, Bacillus subtilis, Salmonella typhimurium, or any bacterial strain capable of expressing a recombinant protein.

Expression constructs can be introduced into the host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Once host cells harbor the expression construct encoding the Cif or Cif fusion protein, the host cells are cultured under culture conditions suitable to express the recombinant protein. The resulting expressed protein can then be purified from either the culture medium or the host cells using known techniques.

In accordance with the production of a Cif fusion, such as those of maltose binding protein, glutathione-S-transferase or thioredoxin, the Cif fusion protein can be isolated by affinity chromatography methods. Kits for expression and purification of such fusion proteins are commercially available from New England BioLab (Beverly, Mass.), Pharmacia (Piscataway, N.J.) and Invitrogen. The protein can also be tagged with an epitope and subsequently purified by using a specific antibody directed to the epitope. One such epitope, FLAG, is commercially available from Kodak as are monoclonal antibodies which recognize the FLAG epitope.

The screening method of the present invention can be carried out using any suitable assay format, e.g., multi-well plates, arrays of Cif protein on plates, and the like, that allows rapid preparation and processing of multiple reactions. Stock solutions of the agents as well as assay components are prepared manually and all subsequent pipetting, diluting, mixing, washing, incubating, sample readout and data collecting can be carried out using commercially available robotic pipetting equipment, automated work stations, and analytical instruments for detecting the output of the assay.

In addition to the reagents provided above, a variety of other reagents can be included in the screening assays of the invention. These include reagents like salts, neutral proteins, e.g., albumin, detergents, etc. Also, reagents that otherwise improve the efficiency of the assay, such as protease inhibitors, nuclease inhibitors, and the like can be used.

Test agents that can be screened in accordance with the methods of the present invention are generally derived from libraries of agents or compounds. Such libraries can contain either collections of pure agents or collections of agent mixtures. Examples of pure agents include, but are not limited to, proteins, antibodies, aptamers, peptides, proteinase inhibitors, nucleic acids, oligonucleotides, iRNA, carbohydrates, lipids, synthetic or semi-synthetic small organic molecules, and purified natural products. Examples of agent mixtures include, but are not limited to, extracts of prokaryotic or eukaryotic cells and tissues, as well as fermentation broths and cell or tissue culture supernates. In the case of agent mixtures, the methods of this invention are not only used to identify those crude mixtures that possess the desired activity, but also provide the means to monitor purification of the active agent from the mixture for characterization and development as a therapeutic drug. In particular, the mixture so identified can be sequentially fractionated by methods commonly known to those skilled in the art which can include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active agent is obtained. In particular embodiments, the test compound is a derivative or analog of tiratricol, e.g., as presented by Formula III herein.

As indicated, the screening method involves contacting Cif protein with a test compound in the presence of CMNGC; and determining whether the test compound inhibits hydrolysis of CMNGC by Cif. The step of determining whether hydrolysis of CMNGC by Cif has been inhibited can be carried out as described herein by detecting the fluorescent signal generated upon hydrolysis of CMNGC. In some embodiments, a test compound is selected as a Cif inhibitor if it decreases the fluorescent signal by, e.g., 10%, 20%, 30%, 40%, 50%, 60%, 70%, 80%, 90%, or 95% as compared to a control, e.g., a Cif protein not contacted with an inhibitor. Inhibitors identified by the instant screening assay find application in inhibiting Cif activity as well as in the amelioration or treatment of one or more of the respiratory diseases described herein.

As demonstrated herein, several classes of compounds were shown to inhibit the hydrolysis of CMNGC by Cif. In particular, it has been shown that tiratricol, and derivatives thereof; urea-based scaffolds; and scaffolds based on human signaling epoxide compounds, e.g., derived from long and very long chain fatty acids can inhibit Cif activity. Accordingly, the present invention also features the use of such compounds, either individually or in combination, for inhibiting the activity of Cif protein and in the treatment of respiratory diseases such as chronic obstructive pulmonary disease, pneumonia and cystic fibrosis or secondary infections thereof.

Compounds within the scope of this invention include those having the structure shown in Formula I.

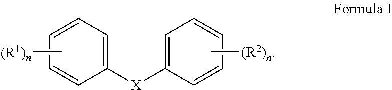

Compounds of Formula I include tiratricol, as well as derivatives and analogs thereof, and urea-based compounds. For the purposes of the present invention, Formula I includes, but is not limited to, biphenyls, diphenylethers, diphenylamines, diphenylmethanes with the substituents of tiratricol, or a diphenyl structure modified to include additional substituents (e.g., O, N, S, OH, CH₃, halo groups, phenyl groups, alkyl groups, etc.), remove substituents (e.g., O, N, S, OH, CH₃, halo groups, phenyl groups, alkyl groups, etc.), or substitute groups (e.g., substitute one halo group for another) in order to provide compounds with improved activity and/or efficacy.

Tiratricol is known in the art as a thyroid hormone analog of use in the treatment of goiter (Alvarez, et al. (2004) Horm. Metab. Res. 36:291-7), as a supplement for weight loss (Devoto & Aravena (2001) Rev. Med. Chil. 129:691-3) and for neutralizing bacterial endotoxins (Cascales, et al. (2008) Chem. Biol. Drug Design 72:320-28).

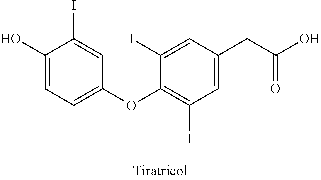 [SMILES: O=C(O)Cc1cc(I)c(Oc2ccc(O)c(I)c2)c(I)c1]

Tiratricol derivatives have been described. For example, GB 803149 teaches compounds having the structure of Formula II,

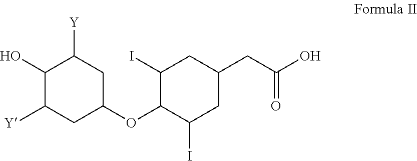

wherein Y is hydrogen or iodine and Y′ is iodine.

Similarly, GB 805761 teaches compounds having the structure of Formula III,

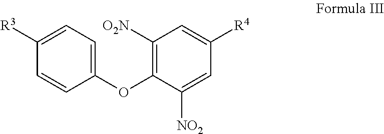

wherein R³ is a hydroxyl group or a group readily convertible thereto and R⁴ is a group readily convertible to an acetic acid side chain.

Thus, in accordance with compounds having the structure of Formula I,

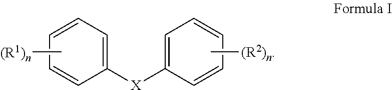

X may be absent or present and when present can be —O—, —NH—, —S—, —CH₂—, —NHC(O)NH—, or —C(O)NHC(O)NH—;

R¹ and R² may be substituted one or more times anywhere on their respective rings,

n is 0 to 5, and

each occurrence of R¹ and R² is independently a hydrogen, hydroxyl, amino, cyano, halo, nitro, mercapto, phosphate, —CH(CH₃)₂—COOH, —CO₂Me, CONH₂, —CONHNH₂, —NHC(O)CH₂COOH, —OCH₂COOH, —OCH(O)CH₂CH₃, alkyl, alkenyl, alkynyl, aryl, or amido group. In certain embodiments, n is at least one.

As used herein, the term “amino” means —NH₂; the term “nitro” means —NO₂; the term “halo” designates —F, —Cl, —Br or —I; the term “cyano” means —CN; and the term “hydroxyl” means —OH.

The term “alkyl” refers to a radical, having a single saturated carbon atom as the point of attachment, no carbon-carbon double or triple bonds, further having a linear or branched, cyclic or acyclic structure. The term alkyl is intended to include substituted and unsubstituted alkyls. Unless otherwise indicated alkyls of the invention have between 1 and 6 carbon atoms.

The term “alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure. The term alkenyl is intended to include substituted and unsubstituted alkenyls. Unless otherwise indicated alkenyls of the invention have between 1 and 6 carbon atoms.

The term “alkynyl” refers to a radical, having a single nonaromatic carbon atom as the point of attachment and at least one nonaromatic carbon-carbon triple bond, further having a linear or branched, cyclic or acyclic structure. The term alkynyl is intended to include substituted and unsubstituted alkynyls. Unless otherwise indicated alkynyls of the invention have between 1 and 6 carbon atoms.

The term “aryl” refers to a radical, having a single carbon atom as point of attachment, wherein the carbon atom is part of an aromatic ring structure containing only carbon atoms. The term aryl is intended to include substituted and unsubstituted aryls. Unless otherwise indicated aryls of the invention have between 5 and 7 carbon atoms.

The term “substituted,” when used to modify a class of organic radicals (e.g., alkyl, aryl, etc.), means that one, or more than one, hydrogen atom of that radical has been replaced by a heteroatom, or a heteroatom containing group. Exemplary substituents include, but are not limited to, oxo, hydroxyl, amino, cyano, halo, nitro, mercapto, phosphate, —COOH, —CO₂Me, CONH₂, —CONHNH₂, or alkyl groups.

The term “unsubstituted,” when used to modify a class of organic radicals (e.g., alkyl, aryl, etc.) means that none of the hydrogen atoms of that radical have been replaced with a heteroatom or a heteroatom containing group.

Monoepoxide derivatives of long chain (13 to 21 carbons) and very long (longer than 22 carbons) chain fatty acids are also of use in inhibiting Cif activity. The epoxyeicosatrienoic acids (all with cis configuration) are produced from arachidonic acid by cytochrome P450 epoxygenase (Zhu, et al. (1995) Hypertension 25:854). Four isomers are formed: 5,6-, 8,9-, 11,12-, and 14,15-EET.

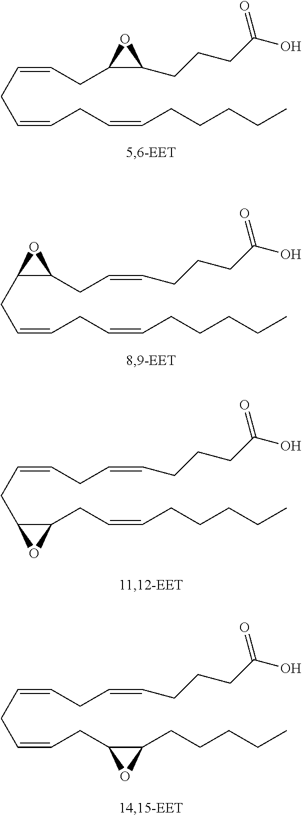 [SMILES: CCCCC/C=C\C/C=C\C/C=C\C[C@H]1O[C@H]1CCCC(=O)O ; CCCCC/C=C\C/C=C\C[C@H]1O[C@H]1C/C=C\CCCC(=O)O ; CCCCC/C=C\C[C@H]1O[C@H]1C/C=C\C/C=C\CCCC(=O)O ; CCCCC[C@H]1O[C@H]1C/C=C\C/C=C\C/C=C\CCCC(=O)O]

In addition, Falck, et al. ((2003) Am. J. Physiol. Heart Circ. Physiol. 284:H337-H349) disclose 19 analogs of 14,15-EET and a series of 14,15-epoxyeicosatrienoyl-sulfonamides are described by Yang, et al. ((2007) J. Pharmacol. Exp. Therapeut. 321:1023-31).

Besides arachidonic acid, epoxide derivatives have been synthesized from EPA (20:5n-3) (Van Rollins (1990) Lipids 25:481) and DHA (22:6n-3) (Van Rollins, et al. (1984) J. Biol. Chem. 259:5776). These derivatives, epoxyeicosaquatraenoic acid (EpEQE) from EPA and epoxydocosapentaenoic acid (EpDPE) are also generated by the action of renal and hepatic cytochrome P-450 monooxygenases (Fer, et al. (2008) Arch. Biochem. Biophys. 471:116). In the rat brain and spinal cord, the regioisomers 17,18-EpEQE and 7,8-EpDPE are the most abundant.

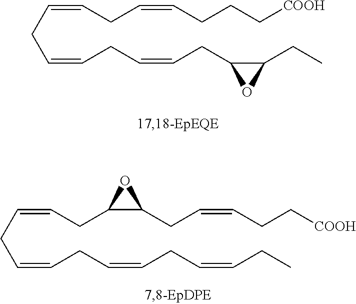 [SMILES: CC[C@H]1O[C@H]1C/C=C\C/C=C\C/C=C\C/C=C\CCCC(=O)O ; CC/C=C\C/C=C\C/C=C\C/C=C\C[C@H]1O[C@H]1C/C=C\CCC(=O)O]

Furthermore, 19,20-EpDPE is a docosahexaenoic acid (DHA) epoxygenase metabolite, derived via epoxidation of the omega-3 double bond of DHA and 17,18-epoxyeicosatetraenoic acid (EpETE) is biosynthesized by stereospecific epoxidation of the omega-3 bond of eicosapentaenoic acid (EPA) (Morisseau, et al. (2010) J. Lipid Res. 51:3481).

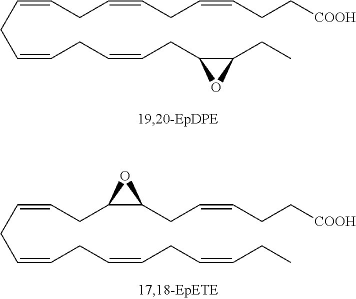 [SMILES: CC[C@H]1O[C@H]1C/C=C\C/C=C\C/C=C\C/C=C\C/C=C\CCC(=O)O ; CC/C=C\C/C=C\C/C=C\C/C=C\C[C@H]1O[C@H]1C/C=C\CCC(=O)O]

Other known fatty acid monoepoxides include, but are not limited to, 9,10-epoxyeicosatetraenoic acid (EpOME)); 12,13-EpOME; α 9,10-epoxyoctadecadienoic acid (EpODE); a 12,13-EpODE; α 15,16-EpODE; 8,9-EpETE; 11,12-EpETE; 14,15-EpETE; 10,11-EpDPE; 13,14-EpDPE; and 16,17-EpDPE (Morisseau, et al. (2010) supra).

Accordingly, in some embodiments, the inhibitory compound of this invention is a long chain or very long chain fatty acid monoepoxide. In particular embodiments, the inhibitor of the invention is a long chain omega-3 fatty acid (e.g., octadecatrienoic acid (ALA), octadecatetraenoic acid, eicosatrienoic acid (ETE), eicosatetraenoic acid (ETA), eicosapentaenoic acid (EPA) or heneicosapentaenoic acid (HPA)); epoxide derivative of a very long chain omega-3 fatty acid (e.g., docosapentaenoic acid (DPA) or docosahexaenoic acid); epoxide derivative of a long chain omega-6 fatty acid (e.g., octadecadienoic acid, eicosadienoic acid, dihomo-gamma-linolenic acid (DLGA) or arachidonic acid (AA)); or epoxide derivative of a very long chain omega-6 fatty acid (e.g., docosadienoic acid, adrenic acid, or docosapentaenoic acid). In particular embodiments, the fatty acid epoxide of the invention is an EET, EpETE or EpDPE. In yet other embodiments, the long chain or very long chain fatty acid monoepoxide is all-cis, all-trans or a mixture and cis and trans fatty acid.

Long or very long chain fatty acid epoxides of this invention can be naturally occurring, synthetically produced or enzymatically produced using an epoxygenase. In addition, the fatty acid epoxides of this invention include methyl esters, ethanolamides, sulfonamides and sulfonimides.

As with the initial screens, the activity of fatty acid epoxides and compounds within the scope of Formula I and can be screened via the assay described herein.

Compounds Formula I or fatty acid epoxides, as well as compounds disclosed in Tables 8, 14 and 15, are Cif inhibitors, which are of use in a method for blocking or inhibiting the activity of Cif. Such a method involves contacting a Cif protein either in vitro or in vivo with an effective amount of a Cif inhibitor so that the activity of the Cif is inhibited or reduced. An effective amount of an inhibitor is an amount that reduces the activity of the Cif by 10%, 20%, 30%, 40%, 50%, 60%, 70%, 80%, 90% or 100%. Such activity can be monitored by enzymatic assays detecting activity of the Cif or by monitoring proteins regulated by Cif (e.g., CFTR or TAP-1).

As indicated, the inhibition of Cif is of use in reducing the ability of P. aeruginosa to infect patient airways; reducing the ability of P. aeruginosa to reverse the effects of CFTR corrector and potentiator compounds in patients with cystic fibrosis; and reducing the ability of P. aeruginosa to shield viral infections from immune surveillance. In addition, P. aeruginosa is also a common pathogen in patients suffering from burns. Therefore, a Cif inhibitor could be useful in burn treatment. Moreover, it has been shown that the Cif from Acinetobacter sp. 13TU RUH2624 can be inhibited by tiratricol. Accordingly, the present invention also features a method for ameliorating or treating a respiratory disease, or a secondary infection thereof, by administering to a subject in need of treatment a pharmaceutical composition containing a compound of Formula I or fatty acid epoxides, or a compound disclosed in Table 8, 14, or 15. In most cases the subject being treated will be a human being, but treatment of agricultural animals, e.g., livestock and poultry, and companion animals, e.g., dogs, cats and horses, is also contemplated. The dosage or effective amount of the Cif inhibitor is an amount which achieves the desired outcome of ameliorating or reducing at least one sign or symptom of a respiratory disease, or a secondary infection thereof.

In particular embodiments of this invention, the respiratory disease being ameliorated or treated is chronic obstructive pulmonary disease, pneumonia, a P. aeruginosa infection, an Acinetobacter infection or cystic fibrosis. As described herein, subjects with CF, COPD or pneumonia exhibit an exacerbation of their respiratory disease when the lungs of said subjects become infected with P. aeruginosa. As such, inhibition of Cif activity in these subjects will result in the amelioration or treatment of the respiratory disease. Furthermore, given its effect on TAP-1, Cif can impede the immune response to viral infections, which are major contributors to clinical exacerbations in patients with cystic fibrosis. Therefore, in other embodiments of the present invention, the Cif inhibitor ameliorates or treats a secondary infection of a subject with a respiratory disease, wherein the secondary infection includes, but is not limited to a viral infection, an Acinetobacter infection or a P. aeruginosa infection.

To evaluate the efficacy of compounds of the invention, one of skill will appreciate that a model system of, e.g., CF with a P. aeruginosa infection, can be utilized to evaluate the adsorption, distribution, metabolism and excretion of a compound as well as its potential toxicity in acute, sub-chronic and chronic studies.

For therapeutic use, it is desirable that the compounds of the present invention are provided to a subject in a pharmaceutically acceptable carrier and at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier, composition or vehicle, is typically a liquid or solid filler, diluent, excipient, or solvent encapsulating material. Each carrier must be acceptable in the sense of being compatible with the other ingredients of the formulation and not injurious to the subject being treated.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In some embodiments, the pharmaceutical composition is appropriately formulated for systemic administration. In other embodiments, the pharmaceutical composition is appropriately formulated for pulmonary administration. For the purposes of the present invention, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli, and the like).

Pulmonary delivery can be achieved by different approaches, including the use of nebulized, aerosolized, micellular and dry powder-based formulations; administration by inhalation may be oral and/or nasal. Delivery can be achieved with liquid nebulizers, aerosol-based inhalers, and dry powder dispersion devices. One of the benefits of using an atomizer or inhaler is that the potential for contamination is minimized because the devices are self-contained. Dry powder dispersion devices, for example, deliver drugs that may be readily formulated as dry powders. A Cif inhibitor composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

Examples of pharmaceutical devices for aerosol delivery include metered dose inhalers (MDIs), dry powder inhalers (DPIs), and air-jet nebulizers. Exemplary delivery systems by inhalation which can be readily adapted for delivery of the instant compound are described in, for example, U.S. Pat. No. 5,756,353; U.S. Pat. No. 5,858,784; WO 98/31346; WO 98/10796; WO 00/27359; WO 01/54664; and WO 02/060412. Other aerosol formulations that may be used for delivering the instant compounds are described in U.S. Pat. No. 6,294,153; U.S. Pat. No. 6,344,194; U.S. Pat. No. 6,071,497; WO 02/066078; WO 02/053190; WO 01/60420; and WO 00/66206.

The invention is described in greater detail by the following non-limiting examples.

### Example 1

### Cif Epoxide Hydrolase Enzyme Activity is Required for CFTR Inhibitory Activity

Mutagenesis and Protein Purification. D129S, E153D, and E153Q mutations were generated using an in vivo yeast recombineering technique as previously described (MacEachran, et al. (2007) supra; Shanks, et al. (2006) Appl. Environ. Microbiol. 72:5027-5036). The H177A, Y239F and H207A mutations were generated by altering the coding sequence of the wild-type Cif expression plasmid pDPM73 (MacEachran, et al. (2007) supra) using the QUIKCHANGE Lightning Site-Directed Mutagenesis Kit (Stratagene). Carboxy-terminal hexa-histidine-tagged Cif protein was expressed in TOP10 Escherichia coli (Invitrogen) cells and purified by immobilized metal affinity chromatography according to established methods (Bahl, et al. (2010) Acta Crystallogr. F66:26-28). Purified Cif protein was prepared in the following buffer: 100 mM NaCl, 20 mM sodium phosphate (pH 7.4).

Crystallization, Data Collection and Processing, Structure Refinement, and Analysis. Cif protein crystals were obtained by vapor diffusion against 400 μl of reservoir solution in a 4 μl hanging drop at 291 K (Bahl, et al. (2010) supra). Drops were set up by mixing the reservoir solution with Cif protein in a 1:1 ratio. Crystallization reservoir solutions are provided in Table 1.

TABLE 1

| | Protein | | | |
|---|---|---|---|---|
| | Amount | PEG 8000 | | Na acetate |
| Mutant | (mg/mL) | (%) | mM CaCl₂ | (mM) |
| Cif-D129S | 5.0 | 16.0 | 125 | 100 |
| Cif-E153D | 5.0 | 14.0 | 125 | 100 |
| Cif-E153Q | 5.0 | 14.0 | 125 | 100 |
| Cif-H177A | 4.5 | 16.0 | 125 | 100 |
| Cif-H207A | 4.5 | 13.0 | 125 | 100 |
| Cif-Y239F | 5.0 | 15.5 | 125 | 100 |

Upon harvesting for data collection, crystals were soaked in a cryoprotectant composed of the reservoir solution supplemented with 20% (wt/vol) glycerol. The crystals were then flash cooled in the nitrogen stream of an Oxford Cryostream 700 operating at 100 K, or by rapid plunging into a liquid nitrogen bath. Oscillation data were collected at 100 K at the X6A beamline of the National Synchrotron Light Source at Brookhaven National Laboratory. Diffraction images were processed and scaled with the XDS package (Kabsch (1993) J. Appl. Cryst. 26:795-800). Molecular replacement and iterative rounds of automated refinement were carried out with PHENIX (Adams, et al. (2010) Acta Crystallogr. D66:213-221; Adams, et al. (2002) Acta Crystallogr. D58:1948-1954). WinCoot (Emsley & Cowtan (2004) Acta Crystallogr. D60:2126-2132) was used for manual adjustment of the model, and PyMOL was used to generate images of the final model.

Determination of Specific Activity. Epoxide hydrolase enzyme activity was determined for the reporter substrate epibromohydrin (Sigma) using an adrenochrome reporter assay as described previously (Bahl, et al., (2010) supra; Cedrone, et al. (2005) Biotechnol. Lett. 27:1921-1927; MacEachran, et al. (2008) Infect. Immun. 76:3197-3206). Lipoprotein lipase from Pseudomonas spp. (Sigma) was used as a negative protein control, and a standard curve was generated using 3-bromo-1,2-propanediol (Sigma). The enzyme reaction was carried out using 20 μM protein with 10 mM substrate incubated at 37° C. for 30 minutes. One unit was defined as 1 μmol of substrate hydrolyzed per minute.

Cell Culture.

Parental human bronchial epithelial CFBE41o-cells stably transduced with CFTR (Bebok, et al. (2005) J. Physiol. 569:601-615) were maintained in minimal essential media (Invitrogen) supplemented with 50 U/ml penicillin (Sigma), 50 μg/ml streptomycin (Sigma), 2 mM L-glutamine (Cellgro), 9.1% [vol/vol] fetal bovine serum, 0.5 μg/ml puromycin (InvivoGen), and 5 μg/ml Plasmocin (InvivoGen) at 37° C. with 5% CO₂. To establish polarized monolayers, 1×10⁶ cells were seeded onto 24 mm TRANSWELL permeable supports (0.4 μm pore size; Corning) and grown in an air-liquid interface culture at 37° C. with 5% CO₂ for 6-9 days prior to use.

Determination of Cell Surface CFTR Levels.

Polarized monolayers of CFBE41o-cells were apically treated with 50 pg of purified Cif protein or a buffer control, and incubated for 1 hour at 37° C. in a 5% CO₂ incubator. The relative abundance of CFTR was then determined by biotinylating all cell surface proteins, capturing the surface pool with immobilized streptavidin resin following cell lysis, and probing for CFTR by western blot as previously described (Bomberger, et al. (2011) Methods Mol. Biol. 741:271-283).

The Acid Nucleophile.

Once a substrate is bound, the first step is a nucleophilic attack. Therefore, the predicted nucleophilic Asp at position 129 was targeted for mutation. A structurally and chemically conservative mutation is desirable to minimize any impact on the protein's structure. An Asp to Asn mutation is the most conservative; however, previous studies have shown that a carboxamide at the nucleophile position of an EH can be hydrolyzed by the enzyme to form a carboxylic acid, thus regenerating a functional, wild-type enzyme active site (Pinot, et al. (1995) supra). Therefore, the nucleophile of Cif was mutated to serine, a non-charged polar amino acid whose incorporation at the nucleophile residue position has been shown to be tolerated for other EHs (Pinot, et al. (1995) supra). Recombinantly expressed Cif-D129S protein was purified from E. coli culture, supernatant using the same protocol as for wild-type. Cif-D129S crystallized using nearly identical conditions as were used for the wild-type protein (Bahl, et al. (2010) supra). Since the conditions under which a protein will crystallize are strongly dependent upon surface contacts, this indicates that there would be few, if any, structural rearrangements due to the D129S mutation. Diffraction data were collected to 1.55 Å resolution, and phase information obtained by molecular replacement with the wild-type Cif structure as a search model. The final refined model displayed excellent agreement with the diffraction data (see Table 2).

TABLE 2

| | Cif-D129S |
|---|---|
| Data Collection | |
| Wavelength (Å) | 0.9782 |
| Space Group | C2 |
| Unit Cell Dimensions | |
| a, b, c (Å) | 168.2, 84.0, 89.2 |
| α, β, γ (°) | 90, 100.4, 90 |
| Resolution (Å) | 46.08-1.55 (1.59-1.55) |
| R_{sym}^{b} (%) | 5.6 (28.4) |
| R_{mrgd-F}^{c} (%) | 7.5 (30.8) |
| I/σ(I) | 16.9 (4.9) |
| Completeness (%) | 98.0 (96.7) |
| Redundancy | 4.2 (4.2) |
| Refinement | |
| Total number of reflections | 173079 |
| Reflections in the test set | 8686 |
| R_{work}^{d}/R_{free}^{e} (%) | 15.9/17.6 |
| Number of atoms: | |
| Protein | 9551 |
| Solvent | 1224 |
| Ligand | 0 |
| Ramachandran plot^{f} (T) | 91.6/8.0/0.4/0 |
| Bₐᵥ (Å²) | |
| Protein | 12.9 |
| Solvent | 26.8 |
| Bond length RMSD | 0.006 |
| Bond angle RMSD | 1.083 |
| PDB ID | 4DLN |

^{a}Values in parentheses are for data in the highest-resolution shell.
^{b}R_{sym} = ΣₕΣᵢ□I(h) − Iᵢ(h)□/ΣₕΣᵢ Iᵢ(h), where Iᵢ(h) and I(h) values are the i-th and mean measurements of the intensity of reflection h.
^{c}R_{mrgd-F} is robust indicator of the agreement of structure factors of symmetry-related reflections and is described by Diederichs & Karplus (1977) Nat. Struct. Biol. 4(4): 269-75.
^{d}R_{work} = Σₕ□F_{obs}(h) − F_{calc}(h)□Σₕ F_{obs}(h), h∈{working set}.
^{e}R_{free} = Σₕ□F_{obs}(h) − F_{calc}(h)□Σₕ F_{obs}(h), h∈{test set}.
^{f}(Core/allowed/generously allowed/disallowed.

A close inspection of the active site demonstrated that a Ser at position 129 could be sterically accommodated. The mutant Ser O_{γ} accepts hydrogen bonds from the backbone amines of Phe63 and Ile130. This feature is similar to the wild-type structure where one O_{γ} of the Asp129 carboxylic acid forms analogous hydrogen bonds, and the other O_{γ} serves as the nucleophile to attack an epoxide substrate. These hydrogen bonds are used to position the side chain of the residue within the active site, and to polarize the Asp carboxylic acid to assist in nucleophile activation. Given that the Cif-D129S mutant positions its side chain pointing toward the protein backbone and away from the substrate, no enzymatic activity was expected. Indeed, enzyme activity was completely abrogated in an assay using the reporter substrate epibromohydrin. Presumably, this mutation should not alter the ability of ring-opening His177 and Tyr239 to bind and coordinate a substrate, nor would it alter the charge relay system. However, by preventing the nucleophilic attack, all detectable enzyme activity was lost.

Furthermore, this effect was not due a conformation change induced by mutation. Alignment of chain A of the Cif-D129S and wild-type models resulted in an RMSD which was lower than the maximum likelihood error estimates for each structure (Table 3). It was therefore concluded that the abrogation of enzyme activity associated with this mutation was solely instigated by the local change in active site functionality.

TABLE 3

| | Maximum-likelihood | |
|---|---|---|
| | based coordinate | |
| | error estimate | RMSD with wild-type Cif |
| | (Å) | (Å²) |
| Cif-D129S | 0.19 | 0.11 |
| Cif-E153D | 0.21 | 0.11 |
| Cif-E153Q | 0.21 | 0.13 |
| Cif-H177A | 0.28 | 0.10 |
| Cif-H207A | 0.21 | 0.12 |
| Cif-Y239F | 0.20 | 0.12 |

The Charge-Relay Acid.

The role of the charge-relay system was subsequently analyzed by mutation of the acid Glu153. Previous studies on the substrate selectivity of Cif have demonstrated that it is an enzyme with unique preferences, although it is most similar to the mammalian EH1 (also known as microsomal EH) (Bahl, et al. (2010) supra). EH1 possesses a catalytic triad, and mutation of the charge relay Glu to Asp resulted in a large increase in the rate of substrate turnover (Arand, et al. (1999) Biochem. J. 337(Pt 1):37-43). While canonical EHs have their charge-relay acid on a loop within the α/β core domain, in Cif this residue is located within a loop that connects the α/β core to the cap domain (Bahl & Madden (2012) supra). This difference alters the directionality of the charge-relay acid, changing the angle and location at which it accepts a hydrogen bond from the catalytic triad His. A detailed structural analysis of the charge-relay acid residue of Cif and other EHs has been described (Bahl & Madden (2012) supra). Although there is currently no structural information available for EH1, sequence alignment clearly reveals that it utilizes a charge-relay acid at the canonical position. In order to thoroughly investigate the charge-relay acid of Cif, the corresponding Glu to Asp mutation was analyzed.

In contrast to EH1, a marked decrease in the specific activity of Cif-E153D was observed. In order to determine if any conformational or structural changes were induced by this mutation, the structure of Cif-E153D was determined (Table 4).

TABLE 4

| | Cif-E153D |
|---|---|
| Data Collection | |
| Wavelength (Å) | 0.9782 |
| Space Group | C2 |
| Unit Cell Dimensions | |
| a, b, c (Å) | 168.4, 84.1, 89.2 |
| α, β, γ (°) | 90, 100.5, 90 |
| Resolution (Å) | 44.07-1.36 (1.45-1.36) |
| R_{sym}^{b} (%) | 7.5 (45.4) |
| R_{mrgd-F}^{c} (%) | 8.5 (39.8) |
| I/σI | 13.6 (3.7) |
| Completeness (%) | 98.7 (97.5) |
| Redundancy | 5.8 (5.8) |
| Refinement | |
| Total number of reflections | 258287 |
| Reflections in the test set | 12908 |
| R_{work}^{d}/R_{free}^{e} (%) | 21.2/22.3 |
| Number of atoms: | |
| Protein | 9451 |
| Solvent | 987 |
| Ligand | 0 |
| Ramachandran plot^{f} (T) | 90.9/8.7/0.4/0 |
| Bₐᵥ (Å²) | |
| Protein | 12.0 |
| Solvent | 22.0 |
| Bond length RMSD | 0.006 |
| Bond angle RMSD | 1.062 |
| PDB ID | 4DM7 |

^{a}Values in parentheses are for data in the highest-resolution shell.
^{b}R_{sym} = ΣₕΣᵢ□I(h) − Iᵢ(h)□/ΣₕΣᵢ Iᵢ(h), where Iᵢ(h) and I(h) values are the i-th and mean measurements of the intensity of reflection h.
^{c}R_{mrgd-F} is robust indicator of the agreement of structure factors of symmetry-related reflections and is described by Diederichs & Karplus (1977) Nat. Struct. Biol. 4(4): 269-75.
^{d}R_{work} = ΣₕF_{obs}(h) − F_{calc}(h)□Σₕ F_{obs}(h), h∈{working set}.
^{e}R_{free} = Σₕ□F_{obs}(h) − F_{calc}(h)□Σₕ F_{obs}(h), h∈{test set 1}.
^{f}Core/allowed/generously allowed/disallowed.

As for Cif-D129S, high quality crystals were generated using conditions similar to those of the wild-type protein, and phase information was obtained by molecular replacement with wild-type Cif as the search model. Upon examination of the refined structure, it was found that no conformational changes were induced upon mutation (Table 3). Asp153 is located at the same position as the wild-type Glu residue, and is also able to form hydrogen bonds with the backbone amides of Gly266 and Met272. However, with a shorter side chain, the Asp is not able to hydrogen bond at the same distances and angles. A finely tuned charge-relay system may require specific interactions between the acid (Glu153) and the base (His297). Perturbation of the acid position would presumably lower the efficacy of this system, which in turn would impair the ability of the enzyme to activate a water molecule to hydrolyze and release the covalent intermediate, thus reducing the specific activity. In the absence of structural information for EH1, it can be speculated that the opposite effects observed by a charge-relay Glu to Asp mutation are somehow due to the positioning of this residue within the protein sequence.

Subsequently, a conservative mutation was introduced to block the function of the charge-relay system of Cif. Gln is polar and sterically similar to the Glu, but does not possess a formal charge. Therefore, a loss of enzyme activity associated with inhibition of the charge-relay system was expected. Indeed, complete abrogation of enzyme activity was observed with Cif-E153Q. Crystallization and structural determination were performed as before (Table, 5), and again no mutation was found to induce conformational differences (Table 3).

TABLE 5

| | Cif-E153Q |
|---|---|
| Data Collection | |
| Wavelength (Å) | 1.0000 |
| Space Group | C2 |
| Unit Cell Dimensions | |
| a, b, c (Å) | 168.4, 83.9, 89.5 |
| α, β, γ (°) | 90, 100.4, 90 |
| Resolution (Å) | 46.13-1.66 (1.70-1.66) |
| R_{sym}^{b} (%) | 9.0 (56.4) |
| R_{mrgd-F}^{c} (%) | 8.4 (38.5) |
| I/σI | 16.5 (4.1) |
| Completeness (%) | 97.1 (95.8) |
| Redundancy | 7.5 (7.5) |
| Refinement | |
| Total number of reflections | 140462 |
| Reflections in the test set | 7020 |
| R_{work}^{d}/R_{free}^{e} (%) | 18.1/20.8 |
| Number of atoms: | |
| Protein | 9496 |
| Solvent | 870 |
| Ligand | 0 |
| Ramachandran plot^{f} (T) | 91.3/8.3/0.4/0 |
| Bₐᵥ (Å²) | |
| Protein | 14.4 |
| Solvent | 24.5 |
| Bond length RMSD | 0.006 |
| Bond angle RMSD | 1.028 |
| PDB ID | 4DMC |

^{a}Values in parentheses are for data in the highest-resolution shell.
^{b}R_{sym} = ΣₕΣᵢ□I(h) − Iᵢ(h)□/ΣₕΣᵢ Iᵢ(h), where Iᵢ(h) and I(h) values are the i-th and mean measurements of the intensity of reflection h.
^{c}R_{mrgd-F} is robust indicator of the agreement of structure factors of symmetry-related reflections and is described by Diederichs & Karplus (1977) Nat. Struct. Biol. 4(4): 269-75.
^{d}R_{work} = Σₕ□F_{obs}(h) − F_{calc}(h)□Σₕ F_{obs}(h), h∈{working set}.
^{e}R_{free} = Σₕ□F_{obs}(h) − F_{calc}(h)□Σₕ F_{obs}(h), h∈{test set}.
^{f}Core/allowed/generously allowed/disallowed.

Upon examination of the Cif-E153Q structure, it was clear that Gln153 was occupying the same position in the active site as the wild-type Glu residue, including the hydrogen bonds to backbone amides. Although the carboxamide orientation could not be determined directly from X-ray diffraction data, one could be assigned based on the hydrogen bonding profile of this residue. Only the carbonyl oxygen was capable of accepting hydrogen bonds from the backbone amides. Therefore, it could be assumed that the amide group was donating a hydrogen bond to the charge-relay His297. In the wild-type protein, the charge-relay acid accepts a hydrogen bond from His297. By reversing the hydrogen bond orientation between the charge-relay acid and base, activation of a water molecule for release of the enzyme-substrate intermediate was blocked. It was important to note that the E153Q mutation was not expected to impair the ability of the ring-opening residues to bind, coordinate, and assist in epoxide ring opening, nor would it impact the nucleophilic attack on the substrate by Asp129. Therefore, it was expected that all substrates would be converted to suicide inhibitors, and covalent adduction would occur in the presence of the E153Q mutation. Observation of a covalent enzyme-substrate intermediate has been previously demonstrated for other EHs by various techniques (Arand, et al. (1996) J. Biol. Chem. 271:4223-4229; Hammock, et al. (1994) Biochem. Biophys. Res. Commun. 198:850-856; Muller, et al. (1997) Eur. J. Biochem. 245:490-496; Pinot, et al. (1995) supra), as well as other α/β hydrolase, Asp nucleophile enzymes (Chan, et al. (2011) J. Am. Chem. Soc. 133:7461-7468; Pieters, et al. (1999) Bioorg. Med. Chem. Lett. 9:161-166).

The Ring-Opening Residues. Further investigation of the Cif active site continued with mutation of ring-opening residues His177 and Tyr239 to Ala and Phe, respectively. These residues are thought to function together for substrate positioning, serve as electron withdrawing groups for the epoxide oxygen, and donate a proton to the epoxide oxygen to form an alcohol upon ring opening. As such, they were analyzed together.

After performing mutagenesis and protein purification for Cif-H177A and Y239F as before, a complete absence of substrate turnover was observed in the presence of either mutation. It was unclear which residue was responsible for donation of a proton to the epoxide oxygen upon ring opening. It was contemplated that H177 was the more likely candidate due to the generally lower pKa of a His side chain compared to a Tyr. It was again confirmed that the mutations were not altering the protein's conformation by employing the same structure determination pipeline for these Cif mutants. In the wild-type structure, it was observed that a water molecule coordinated by His177 and Tyr239 presumably occupies the position of a substrate epoxide oxygen. It was interesting that this water molecule was still present in the Y239F structure, but was absent with the H177A mutation. These structures were determined to similar resolution (Table 6), so the reason for this was unclear.

TABLE 6

| | |
|---|---|
| Cif-H177A | Cif-Y239F |

| | | |
|---|---|---|
| Data Collection | | |
| Wavelength (Å) | 0.9770 | 0.9770 |
| Space Group | C2 | C2 |
| Unit Cell Dimensions | | |
| a, b, c (Å) | 167.8, 83.8, 89.0 | 168.7, 84.0, 89.3 |
| α, β, γ (°) | 90, 100.4, 90 | 90, 100.5, 90 |
| Resolution (Å) | 45.98-2.12 | 46.18-1.5 |
| | (2.20-2.12) | (1.61-1.50) |
| R_{sym}^{b} (%) | 9.7 (38.2) | 5.8 (30.2) |
| R_{mrgd-F}^{c} (%) | 14.1 (39.5) | 8.5 (33.4) |
| I/σI | 12.7 (4.0) | 16.1 (4.8) |
| Completeness (%) | 99.8 (99.7) | 97.2 (95.8) |
| Redundancy | 4.2 (4.2) | 4.3 (4.3) |
| Refinement | | |
| Total number of reflections | 68795 | 190239 |
| Reflections in the test set | 3454 | 9495 |
| R_{work}^{d}/R_{free}^{e} (%) | 16.6/20.6 | 16.3/18.2 |
| Number of atoms: | | |
| Protein | 9340 | 9566 |
| Solvent | 555 | 1250 |
| Ligand | 0 | 24 |
| Ramachandran plot^{f} (T) | 91.3/8.3/0.4/0 | 91.3/8.3/0.4/0 |
| Bₐᵥ (Å²) | | |
| Protein | 19.0 | 12.3 |
| Solvent | 26.7 | 25.8 |
| Bond length RMSD | 0.007 | 0.006 |
| Bond angle RMSD | 0.994 | 1.069 |
| PDB ID | 4DMF | 4DMK |

^{a}Values in parentheses are for data in the highest-resolution shell.
^{b}R_{sym} = ΣₕΣᵢ□I(h) − Iᵢ(h)□/ΣₕΣᵢ Iᵢ(h), where Iᵢ(h) and I(h) values are the i-th and mean measurements of the intensity of reflection h.
^{c}R_{mrgd-F} is robust indicator of the agreement of structure factors of symmetry-related reflections and is described by Diederichs & Karplus (1977) Nat. Struct. Biol. 4(4): 269-75.
^{d}R_{work} = Σₕ□F_{obs}(h) − F_{calc}(h)□Σₕ F_{obs}(h), h∈{working set}.
^{e}R_{free} = Σₕ□F_{obs}(h) − F_{calc}(h)□Σₕ F_{obs}(h), h∈{test set}.
^{f}Core/allowed/generously allowed/disallowed.

The ring opening pair was also responsible for correctly positioning a substrate for nucleophilic attack. Previous analysis of the Cif-H177Y mutation showed that when the substrate binding position was altered, all EH activity was lost (Bahl & Madden (2012) supra). Taken together, without the ability to bind and coordinate a substrate, or the ability to assist in ring opening, EH activity is inhibited by these mutations.

The Substrate Tunnel His.

The final residue investigated was His207. The sterically defined entrance to the active site of Cif is a funnel lined with a succession of His residues, beginning with ring opening His177, followed by His207, and terminating at the protein surface with His269. To investigate the role of His207 in the catalytic mechanism of Cif, an Ala mutation was generated. Purification of this mutant yielded a 2-fold reduction in the amount of protein produced. Upon assay for epibromohydrin hydrolysis, it was found that the H207A mutation greatly impaired the ability of Cif to function as an EH. However, unlike the residues that directly play a role in catalysis, this mutation did not abrogate all enzyme activity. To examine any conformational impact of this mutation, the structure of Cif-H207A was determined (Table 7), and, again no difference in the protein conformation was found.

TABLE 7

| | Cif-H207A |
|---|---|
| Data Collection | |
| Wavelength (Å) | 0.9770 |
| Space Group | C2 |
| Unit Cell Dimensions | |
| a, b, c (Å) | 168.8, 83.8, 89.5 |
| α, β, γ (°) | 90, 100.5, 90 |
| Resolution (Å) | 46.18-1.90 (1.95-1.90) |
| R_{sym}^{b} (%) | 7.0 (33.6) |
| R_{mrgd-F}^{c} (%) | 9.4 (35.4) |
| I/σI | 16.3 (4.4) |
| Completeness (%) | 97.3 (96.3) |
| Redundancy | 4.3 (4.3) |
| Refinement | |
| Total number of reflections | 94109 |
| Reflections in the test set | 4706 |
| R_{work}^{d}/R_{free}^{e} (%) | 16.2/19.8 |
| Number of atoms: | |
| Protein | 9491 |
| Solvent | 824 |
| Ligand | 24 |
| Ramachandran plot^{f} (T) | 91.2/8.4/0.4/0 |
| Bₐᵥ (Å²) | |
| Protein | 15.5 |
| Solvent | 25.6 |
| Bond length RMSD | 0.006 |
| Bond angle RMSD | 1.007 |
| PDB ID | 4DMH |

^{a}Values in parentheses are for data in the highest-resolution shell.
^{b}R_{sym} = ΣₕΣᵢ□I(h) − Iᵢ(h)□/ΣₕΣᵢ Iᵢ(h), where Iᵢ(h) and I(h) values are the i-th and mean measurements of the intensity of reflection h.
^{c}R_{mrgd-F} is robust indicator of the agreement of structure factors of symmetry-related reflections and is described by Diederichs & Karplus (1977) Nat. Struct. Biol. 4(4): 269-75.
^{d}R_{work} = Σₕ□F_{obs}(h) − F_{calc}(h)□Σₕ F_{obs}(h), h∈{working set}.
^{e}R_{free} = Σₕ□F_{obs}(h) − F_{calc}(h)□Σₕ F_{obs}(h), h∈{test set}.
^{f}Core/allowed/generously allowed/disallowed.

It was interesting to note that although His207 clearly contributed to epoxide hydrolysis in Cif, it was not conserved with any other known EH. This suggests two possible roles for this residue, highlighted by features unique to the Cif EH. As previously discussed (Bahl & Madden (2012) supra; Bahl, et al. (2010) supra), the active site of Cif is contained at the end of a pocket within the protein. The first likely role of His207 is to interact with the substrate as it traverses the tunnel through the protein to the active site pocket, and its mutation could therefore limit substrate access to the catalytic residues. Secondly, Cif utilizes a His at position 177 as one of the ring-opening residues in place of a canonical Tyr found in all other EHs (Bahl & Madden (2012) supra). Although there do not appear to be any direct hydrogen bond networks between His207 and His177, their close proximity to one another suggests the H207A mutation could be affecting the local pH. Therefore, the H207A mutation could impede the hydrogen bond or proton donating ability of His177, which would result in the observed decrease in epibromohydrin hydrolysis observed with this mutant. These two proposed functions for His207 are not mutually exclusive, as this residue could be contributing to epoxide hydrolysis in multiple ways. However, it is important to note that while His207 is clearly involved in the catalytic mechanism of Cif, a definitive role for this residue cannot be determined from the data presented here.

Additional Observations from the Multiple Cif Mutant Structures.

With such a large array of mutant Cif protein structures, a few additional observations were noted. In the structures of Cif-Y239F and H207A, a glycerol molecule was found coordinated within each active site. Interestingly, clear electron density corresponding to glycerol molecules was note found in any other Cif structure. Additionally, these glycerol molecules were not coordinated at the same position in the different mutants, and there was even some variability across the asymmetric unit of a single mutant. For Cif-Y239F, glycerol was predominantly occupying an open space within the active site pocket, not likely to reflect a catalytically relevant substrate binding site. Alternatively, glycerol was coordinated by the ring-opening residues His177 and Tyr239 in the Cif-H207A structure. This likely reflects the position occupied by a vicinal diol hydrolysis product. Observation of these glycerol molecules illuminates the impact subtle mutations can have on the stereo-selectivity and accessibility of the active site to small molecules.

Another feature discovered was a small shift in the loop containing the β7 hairpin. This results in displacement at the C_{α} position of residues 170-172, with the maximal shift of ≈5 Å occurring at Gly171. In many of the structures, partial density for an alternate conformation was observed. However, this was not of sufficient quality to model accurately. For others, either the alternate conformation alone, or both were observed. Interestingly, this difference only occurred in one protomer of the Cif dimer. The Cif dimer was not perfectly symmetrical, and the loop shift was only observed with equivalent chains A and C of the asymmetric unit. All of the engineered mutations occurred a minimum distance of ≈8 Å away from this loop, and no residues in between this loop and the site of mutation appeared to shift in any significant way. Therefore, a direct correlation between β7 hairpin loop motion and mutation was not detected. If this conformational sampling were simply stochastic, both conformations in all structures would be expected. It is contemplated that some mutations cause subtle, long range electrostatic effects, and this in turn alters the equilibrium between the two β7 hairpin conformations. Why this only occurs in one protomer of the Cif dimer remains unclear, although it suggests that the alternate conformation is not strongly favored thermodynamically.

Enzyme Activity is Required for the Cellular Effects of Cif.

To examine the relationship between Cif's EH activity and its function as a virulence factor, the panel of mutants were tested for the ability to promote the removal of CFTR from the apical membrane of human airway epithelial cells. Cell surface CFTR abundance was determined by biotinylating all cell surface proteins, capturing this pool with streptavidin, and probing for CFTR via semi-quantitative western blot. It was found that EH enzyme activity was strictly required for the Cif effect. Therefore, it was a logical conclusion that an endogenous human epoxide substrate was also required. It was interesting to note that Cif-E153D and H207A retained some residual levels of enzyme activity, yet they did not exhibit any ability to promote removal of CFTR from the apical membrane of human cells. Either fully wild-type levels of enzyme activity are required for this effect, or these mutations may alter the substrate selectivity of the enzyme and block Cif-mediated hydrolysis of a cellular substrate.

Many human epoxides are potent signaling molecules (Chiamvimonvat, et al. (2007) J. Cardiovasc. Pharmacol. 50:225-237; Spector (2009) J. Lipid Res. 50 Suppl:S52-56), and therefore the absence of an epoxide could be mediating the effect rather than the generation of a hydrolysis product. Cif-E153Q retained an active nucleophile and was able to attack a substrate, removing it from the cellular pool. However, this can affect only a very small quantity of substrate, as Cif-E153Q will sequester an epoxide substrate stiochiometrically. While a slight decrease in apical CFTR levels were observed with this mutation, the effect was not statistically significant (P=0.1).

### Example 2

### Identification and Characterization of an Epoxide Hydrolase Inhibitor for P. aeruginosa Cif

Generation of a CifR Expression Plasmid. The cifR sequence, from locus PA14_{—}26140, was amplified from P. aeruginosa UCBPP-PA14 genomic DNA by PCR using PHUSION high fidelity polymerase (Finnzymes) with the following primers: BspHI_CifR_F (5′-tat atc atg aca acg cga ggc agg cca cgg-3′; SEQ ID NO:1) and BspHI_CifR_R (5′-ggt agt cat gat ggg gcc ctg gaa gag cac ctc cag ggg cca ggc gcg cag cgc ccg tt-3′; SEQ ID NO:2). The PCR product was digested with BspHI and ligated with T4 ligase (NEB) into an NcoI-digested and phosphatase-treated pET16b vector (Novagen). Ligated plasmid was transformed into TOP10 E. coli (Invitrogen) and transformants were selected by growth at 37° C. on LB media supplemented with 150 μg/ml ampicillin. Positive clones were verified by DNA sequence analysis. This generated a CifR construct that possesses a carboxy-terminal deca-histidine tag, preceded by a cleavage site for human rhinovirus 3C(HRV-3C) protease: LEVLFQGP (SEQ ID NO:3).

Protein Purification.

Carboxy-terminal hexa-histidine-tagged Cif protein was expressed in TOP10 E. coli (Invitrogen) cells and purified by immobilized metal affinity chromatography (IMAC) as described previously (Bahl, et al. (2010) supra). Purified Cif protein was prepared in the following buffer: 100 mM NaCl, 20 mM sodium phosphate (pH 7.4).

CifR protein was also purified by IMAC, with subsequent removal of the histidine affinity tag. ROSETTA 2 DE3 (Novagen) E. coli transformed with the CifR expression plasmid were grown in 4 L of 2×YT broth supplemented with 100 μg/ml ampicillin and 34 μg/ml chloramphenicol at 37° C. Expression of CifR protein was then induced at OD₆₀₀=0.6 by addition of isopropyl-β-D-thiogalactopyranoside to 100 μM, and cultures were incubated overnight at 16° C. Cells were harvested from the medium by centrifugation at 5,000 g for 15 minutes at 4° C. Following removal of supernatant, cell pellets were re-suspended in 25 mL of lysis buffer per 1 L of culture volume. The lysis buffer was composed of: 50 mM Tris pH 8.5, 150 mM NaCl, 2 mM MgCl₂, 1 mM ATP, 25 units/mL BENZONASE nuclease (Novagen), and 1 EDTA-free Complete Protease Inhibitor Cocktail Tablet (Roche) per 50 mL. Cells were lysed using a French Press, and the lysate clarified by centrifugation at 40,000 RPM for 1 hour at 4° C. in a Ti45 rotor (Beckman). Supernatant was then passed over a 5 mL column of Ni SEPHAROSE resin (GE Healthcare) that had been pre-equilibrated with IMAC buffer composed of: 50 mM Tris pH 8.5, 500 mM NaCl, and 1 mM dithiothreitol (DTT) with 20 mM imidazole pH 8.5. Following a wash with 10 column volumes of IMAC buffer containing 77 mM imidazole to remove unbound material, CifR protein was eluted from the resin over a 15 column volume gradient running from 248 mM to 324 mM imidazole in IMAC buffer. Fractions were pooled, concentrated, and dialyzed into gel filtration buffer containing: 25 mM Tris pH 8.5, 150 mM NaCl, 0.1 mM DTT, and 0.1 mM ATP. The protein concentration was determined by Bradford assay (Biorad), and HRV-3C protease was added to a mass ratio of 1:10 (protease:CifR). Cleavage of the deca-histidine tag proceeded overnight at 4° C. The HRV-3C protease possesses a non-cleavable histidine tag, which is subsequently used to remove it, along with any uncleaved CifR protein, from the sample by passing over a 5 mL column of Ni SEPHAROSE resin (GE). The flow-through was collected, and mature CifR protein was further clarified by size-exclusion chromatography with a HILOAD SUPERDEX 200 prep grade 26/60 column (GE Healthcare) using gel filtration buffer. Purified, matured CifR protein was prepared in: 10 mM Tris pH 8.5, 50 mM KCl, 1 mM DTT, and 5 mM MgCl₂.

Epoxide Hydrolase Enzyme Assay.

The radioactivity assay was carried out using tritium-labeled cis-stilbene oxide (CSO) as the substrate as described previously (Gill, et al. (1983) Anal. Biochem. 131:273-82).

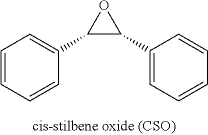 [SMILES: c1ccc([C@H]2O[C@H]2c2ccccc2)cc1]

The adrenochrome reporter assay was performed as described previously (Bahl, et al. (2010) supra; MacEachran, et al. (2008) supra; Cedrone, et al. (2005) supra) using 1,2-epoxyhexane (Sigma) as the substrate.

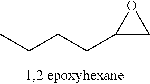

Assays with fluorogenic reporter substrates were carried out as described (Jones, et al. (2005) Anal. Biochem. 343:66-75; Morisseau, et al. (2011) Anal. Biochem. 414:154-62). For assays using Cyano(6-methoxynaphthalen-2-yl)methyl (oxiran-2-ylmethyl) (CMNGC) as the fluorogenic substrate, measurements were taking using a fluorescent plate reader with λₑₓ=330 nm and λₑₘ=465 nm.

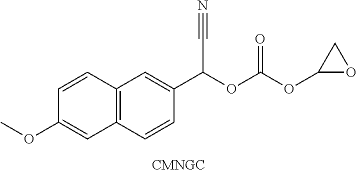 [SMILES: COc1ccc2cc(C(C#N)OC(=O)OC3CO3)ccc2c1]

High Throughput Screening. All high throughput screening assays were performed using 96-well plates, a 200 μL reaction volume, and a fluorescent plate reader. The primary screen was carried out using 25 μM CMNGC as the fluorogenic reporter substrate, 1 μM Cif protein, test compounds at 10 μM, and 10 μM N,N′-di-(3,4-dichlorophenyl) urea as a positive control for Cif inhibition. The fluorescent signal was measured after allowing the enzyme reaction to proceed for 60 minutes. The secondary screen was performed using the same conditions as the primary screen, except that fluorescent readings were taken every minute for the 60-minute reaction.

Crystallization, Data Collection and Processing, Structure Refinement, and Analysis.

Cif-tiratricol co-crystals were obtained by vapor diffusion against 400 μl of reservoir solution in a 4 μl hanging drop at 291 K (Bahl, et al. (2010) supra). A solution of 8.1 mg/ml Cif protein containing 200 μM tiratricol was mixed in a 1:1 ratio with reservoir solution consisting of 15% (wt/vol) polyethylene glycol 8000, 125 mM CaCl₂, 100 mM sodium acetate (pH 5), 200 μM tiratricol, 0.2% (vol/vol) dimethyl sulfoxide (DMSO). Prior to data collection, crystals were washed in cryo solution composed of 15% (wt/vol) polyethylene glycol 8000, 125 mM CaCl₂, 100 mM sodium acetate (pH 5), 200 μM tiratricol, 0.2% (vol/vol) DMSO, 20% (wt/vol) glycerol and flash-cooled by plunging into a liquid nitrogen bath. Oscillation data were collected at 100 K at the X6A beamline of the National Synchrotron Light Source at Brookhaven National Laboratory. Diffraction images were processed and scaled with the XDS package (Kabsch (1993) J. Appl. Cryst. 26:795-800). Molecular replacement and iterative rounds of automated refinement were carried out with PHENIX (Adams, et al. (2010) supra; Adams, et al. (2002) supra). WinCoot (Emsley & Cowtan (2004) supra) was used for manual adjustment of the model, and PyMOL was used to generate images of the final model.

Discovery of a First Generation Cif Inhibitor.

The initial screen employed a manually chosen collection of 16 compounds, and enzyme activity of Cif was determined by the radioactivity assay (Table 8). The most promising compounds approached 18% inhibition. The top seven compounds were retested and two chemically related compounds added (Table 8). From the secondary screen, the most potent inhibition was found with N,N′-di-(3,4-dichlorophenyl) urea (DCPU, Compound 18), a compound that inhibited 25%±3% (P=0.004) of Cif enzyme activity.

TABLE 8

| Compound | | Inhibition |
|---|---|---|
| No. | Chemical Structure | (%) |
| 1^{a} | 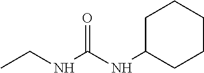 | ~2.5 |
| 2 | 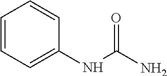 | 0 |
| 3 | 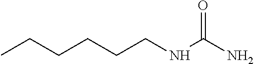 | 3 |
| 4 | 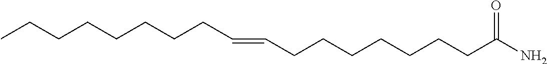 [SMILES: CCCCCCCC/C=C/CCCCCCCC(N)=O] | 0 |
| 5 | 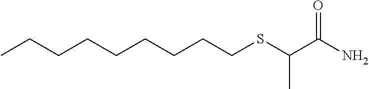 | 8 |
| 6^{b} | 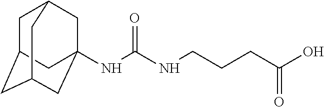 [SMILES: O=C(O)CCCNC(=O)NC12CC3CC(CC(C3)C1)C2] | 0 |
| 7^{a} | 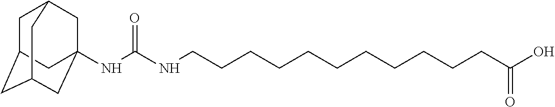 [SMILES: O=C(O)CCCCCCCCCCCNC(=O)NC12CC3CC(CC(C3)C1)C2] | 3 |
| 8 | 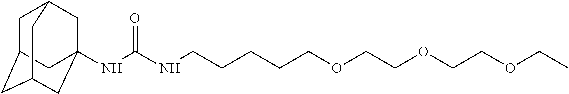 [SMILES: CCOCCOCCOCCCCCNC(=O)NC12CC3CC(CC(C3)C1)C2] | 2.5 |
| 9 | 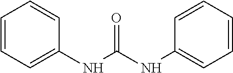 [SMILES: O=C(Nc1ccccc1)Nc1ccccc1] | 13 |
| 10 | 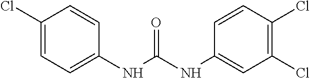 [SMILES: O=C(Nc1ccc(Cl)cc1)Nc1ccc(Cl)c(Cl)c1] | 13.5 |
| 11 | 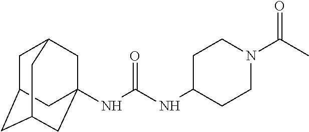 [SMILES: CC(=O)N1CCC(NC(=O)NC23CC4CC(CC(C4)C2)C3)CC1] | 2 |
| 12 | 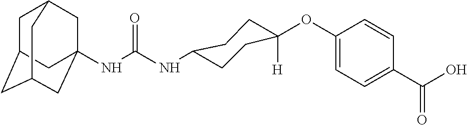 [SMILES: O=C(NC1CCC(Oc2ccc(C(=O)O)cc2)CC1)NC12CC3CC(CC(C3)C1)C2] | 14 |
| 13 | 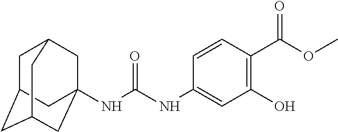 [SMILES: COC(=O)c1ccc(NC(=O)NC23CC4CC(CC(C4)C2)C3)cc1O] | 18 |
| 14 | 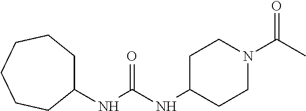 [SMILES: CC(=O)N1CCC(NC(=O)NC2CCCCCC2)CC1] | 6 |
| 15 | 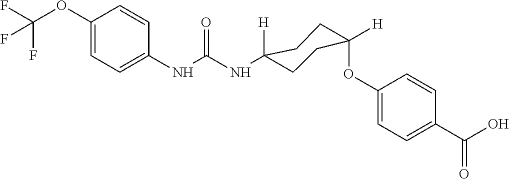 [SMILES: O=C(Nc1ccc(OC(F)(F)F)cc1)NC1CCC(Oc2ccc(C(=O)O)cc2)CC1] | 17 |
| 16 | 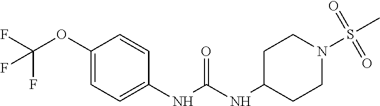 [SMILES: CS(=O)(=O)N1CCC(NC(=O)Nc2ccc(OC(F)(F)F)cc2)CC1] | 14 |
| 17 | 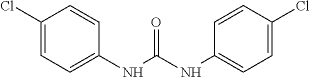 [SMILES: O=C(Nc1ccc(Cl)cc1)Nc1ccc(Cl)cc1] | 7 |
| 18 | 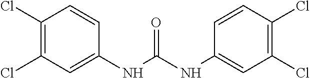 [SMILES: O=C(Nc1ccc(Cl)c(Cl)c1)Nc1ccc(Cl)c(Cl)c1] | 25.5 |

^{a}Wolf, et al. (2006) Anal. Biochem. 355:71-80.
^{b}Gomez, et al. (2006) Protein Sci. 15:58-64.

While a compound with detectable inhibition of Cif EH activity was identified out of a minimal library of compounds, this effect was nominal. Attempts to co-crystallize this compound with Cif protein to determine the mechanism of inhibition were unsuccessful, likely due to the poor solubility and weak binding of DCPU to Cif. With a first-hit molecule identified, the next step was to discover a more potent inhibitor. This required significantly expanding the small molecule search space. The radioactivity assay, while useful on a small-scale, was not amenable to large-scale high throughput screening. CSO is a poor substrate for Cif (Bahl, et al. (2010) supra), the assay generates radioactive waste, and the organic extraction step impedes robotic automation. Since no current assay for Cif enzyme activity was compatible with high throughput analysis, a new assay was required.

Design of a High Throughput Enzyme Assay for Cif.

Fluorogenic compounds for mammalian EH2 (also known as soluble EH; Jones, et al. (2005) supra) have previously been used for high throughput screening for enzyme inhibitors (Xie, et al. (2009) Bioorg. Med. Chem. Lett. 19:2354-9). These compounds contain an epoxide moiety adjacent to an ester, which in turn is linked to a proto-fluorophore. Upon epoxide hydrolysis, the newly generated vicinal-diol undergoes intramolecular cyclization with the adjacent ester, cleaving off a compound that quickly decays to produce a fluorophore (Jones, et al. (2005) supra). Cif was tested for its ability to cleave the EH2 fluorogenic substrates; however, a fluorescent signal above the background was not detectable.

As an EH, Cif possesses a unique substrate selectivity profile. Based on previous work, Cif appears to prefer terminal, generally mono-substituted epoxide substrates (Bahl, et al. (2010) supra). While CSO is disubstituted, the epoxide moiety is physically located at the end of the molecule due to the cis bond. Existing EH2 fluorogenic substrates possess di-substituted epoxide moieties, and may be incompatible with Cif for this reason. To investigate if Cif can hydrolyze the epoxy-ester portion of a fluorogenic compound where the epoxide is mono-substituted, hydrolysis of glycidyl methacrylate and allyl glycidyl ether using the adrenochrome reporter assay was determined. Cif was capable of hydrolyzing these substrates, and therefore seemed likely be able to hydrolyze a fluorogenic epoxide substrate with a terminal epoxide molecule. In parallel, a series of fluorogenic epoxide substrates were designed to possess a mono-substituted epoxide moiety, which have proven useful with mammalian EH1 (also known as microsomal EH; Morisseau, et al. (2011) supra). Therefore, Cif was examined for the ability to catalyze their hydrolysis. It was found that Cif could act on each of these compounds, and optimal signal was achieved using CMNGC. Additionally, the fluorescent signal was completely lost in the presence of the D129S mutation, which was shown herein to abrogate all enzyme activity of Cif. Assay conditions were then optimized for high throughput screening.

High Throughput Screening for Cif Inhibitors.

Toxicity is often a limiting factor when developing small molecules for clinical use. While no toxicology has yet been performed on DCPU, as a polychlorinated aromatic it is unlikely to be well-tolerated by mammalian cells. For a second generation Cif inhibitor, a molecule with limited toxicity and the potential for immediate clinical application was sought. A library of 1600 FDA-approved compounds was screened for Cif inhibitory activity. Upon performing the primary screen, low signal-to-background and high signal-to-noise ratios were observed. In addition, excellent Z′ values were found using DCPU as a positive control for inhibition. Taken together with the low intraplate variability of the positive control samples, these metrics indicate a robust assay for Cif inhibition (Table 9). These statistical parameters are commonly used to evaluate high throughput screening assays, and have been described in detail (Zhang, et al. (1999) J. Biomol. Screen 4:67-73).

TABLE 9

| | Total Activity | | | Positive Control | | |
|---|---|---|---|---|---|---|
| | Control | | | Inhibitor | | |
| Plate# | S/B^{a} | S/N^{b} | Z′^{c} | % I^{d} | SD^{e} | Z^{c} |
| 1 | 3.1 | 60.2 | 0.68 | 20.9 | 1.3 | 0.71 |
| 2 | 2.9 | 99.8 | 0.75 | 30.0 | 0.5 | 0.86 |
| 3 | 3.0 | 44.4 | 0.76 | 14.0 | 0.3 | 0.77 |
| 4 | 2.9 | 78.5 | 0.78 | 15.9 | 0.8 | 0.74 |
| 5 | 2.8 | 65.3 | 0.72 | 14.6 | 0.7 | 0.78 |
| 6 | 2.9 | 71.4 | 0.83 | 30.0 | 1.1 | 0.79 |
| 7 | 3.0 | 48.3 | 0.82 | 19.8 | 0.5 | 0.74 |
| 8 | 2.9 | 62.6 | 0.80 | 26.0 | 1.1 | 0.72 |
| 9 | 3.0 | 54.1 | 0.87 | 15.7 | 0.2 | 0.82 |
| 10 | 2.9 | 138.2 | 0.79 | 20.7 | 0.7 | 0.78 |
| 11 | 2.9 | 123.1 | 0.86 | 24.8 | 0.6 | 0.87 |
| 12 | 2.8 | 145.3 | 0.83 | 19.0 | 0.7 | 0.83 |
| 13 | 2.9 | 59.7 | 0.87 | 15.2 | 0.2 | 0.84 |
| 14 | 2.9 | 143.3 | 0.89 | 17.9 | 0.4 | 0.86 |
| 15 | 2.9 | 49.1 | 0.87 | 25.0 | 1.7 | 0.70 |
| 16 | 3.1 | 79.1 | 0.86 | 15.2 | 0.5 | 0.75 |
| 17 | 2.9 | 94.8 | 0.80 | 24.3 | 0.9 | 0.79 |
| 18 | 2.9 | 89.7 | 0.88 | 17.3 | 0.6 | 0.75 |
| 19 | 2.9 | 81.7 | 0.91 | 17.2 | 0.5 | 0.82 |
| 20 | 2.9 | 69.5 | 0.91 | 20.1 | 0.6 | 0.83 |
| Average | 2.9 | 83 | 0.82 | 20 | 0.7 | 0.79 |
| SD^{e} | 0.1 | 32 | 0.06 | 5 | | 0.05 |

Statistical parameters utilized herein are described in detail by Zhang et al. (1999) supra.
^{a}S/B = signal-to-background ratio.
^{b}S/N = signal-to-noise ratio.
^{c}Z′ is a statistical parameter indicative of assay robustness and reproducibility.
^{d}% I = percent of the total enzyme activity inhibited.
^{e}SD = standard deviation.
^{f}Z = screening window coefficient.

The screen was performed with only a 10-fold stoichiometric excess of inhibitor over Cif enzyme. Therefore, the threshold for a positive hit was set at the relatively low value of 30% inhibition. Using this criterion 47 compounds were identified, yielding a 2.9% hit rate (Table 10).

TABLE 10

| Plate | % Inhibition | Compound Name |
|---|---|---|
| 1 | 48 | Mefenamic acid |
| 2 | 31 | Enalapril maleate |
| 2 | 34 | Ketoprofen |
| 2 | 32 | Cefoxitin sodium |
| 3 | 36 | Canrenone |
| 4 | 86 | Benserazide hydrochloride |
| 5 | 71 | Equilin |
| 5 | 39 | Estradiol |
| 6 | 32 | Eugenol |
| 6 | 31 | Fluorometholone |
| 6 | 48 | Ibuprofen |
| 6 | 32 | Mercaptopurine |
| 7 | 42 | Sulfadiazine |
| 7 | 40 | Tobramycin |
| 8 | 35 | Triamterene |
| 8 | 32 | Warfarin |
| 9 | 33 | Betaine hydrochloride |
| 9 | 33 | Triflupromazine hydrochloride |
| 11 | 32 | Flunixin meglumine |
| 11 | 47 | Acarbose |
| 11 | 79 | Azaperone |
| 11 | 48 | Azelastine hydrochloride |
| 12 | 31 | Toremiphene citrate |
| 12 | 34 | Pregnenolone succinate |
| 12 | 66 | Desoxymetasone |
| 13 | 33 | Buspirone hydrochloride |
| 14 | 52 | Ethanolamine oleate |
| 14 | 36 | Dimethyl fumarate |
| 14 | 34 | Modaline sulfate |
| 14 | 46 | Thiostrepton |
| 14 | 51 | Clomipramine hydrochloride |
| 14 | 36 | Tilorone |
| 14 | 35 | Saxagliptin |
| 15 | 34 | Gadoteridol |
| 15 | 43 | Clofarabine |
| 15 | 35 | Vorinostat |
| 15 | 38 | Algestone acetophenide |
| 15 | 45 | Penciclovir |
| 16 | 41 | Candicidin |
| 18 | 46 | Protoporphyrin ix |
| 18 | 42 | Octopamine hydrochloride |
| 19 | 86 | Tiratricol |
| 19 | 43 | Edaravone |
| 20 | 78 | Sodium tetradecyl sulfate |
| 20 | 47 | Merbromin |
| 20 | 44 | Cetrimonium bromide |
| 20 | 34 | Miltefosine |

Four of the compounds identified in the primary screen were shown to quench the fluorescent signal generated by the assay, and thus were not selected for further evaluation. Subsequently, the filtered primary hits were retested to validate the observed inhibitory effect. After performing the secondary screen in sextuplicate (Table 11), robust indicators of assay quality were again observed (Table 12).

TABLE 11

| | % Inhibition | | | | | | |
|---|---|---|---|---|---|---|---|
| | | | | | | | Mean ± |
| Compound Name | 1* | 2* | 3* | 4* | 5* | 6* | SD |
| Mefenamic acid | 5 | 24 | 17 | 22 | 20 | 6 | 16 ± 8 |
| Enalapril maleate | −6 | 13 | 1 | 9 | 10 | −3 | 4 ± 8 |
| Ketoprofen | 2 | 14 | 9 | 8 | 15 | 4 | 9 ± 5 |
| Cefoxitin sodium | −3 | 16 | 5 | 9 | 19 | 14 | 10 ± 8 |
| Canrenone | 16 | 27 | 20 | 24 | 28 | 27 | 24 ± 5 |
| Benserazide | 77 | 85 | 80 | 74 | 78 | 80 | 79 ± 4 |
| hydrochloride | | | | | | | |
| Estradiol | 4 | 5 | 5 | 6 | 8 | −6 | 4 ± 5 |
| Eugenol | 4 | 0 | 7 | 4 | 6 | −4 | 3 ± 4 |
| Fluorometholone | 18 | 15 | 19 | 16 | 21 | 16 | 18 ± 2 |
| Ibuprofen | 1 | 6 | 5 | 8 | 15 | 7 | 7 ± 5 |
| Mercaptopurine | 5 | 15 | 19 | 15 | 15 | 11 | 13 ± 5 |
| Sulfadiazine | 23 | 24 | 24 | 26 | 28 | 15 | 23 ± 4 |
| Tobramycin | −5 | −7 | −5 | −12 | 0 | −21 | −8 ± 7 |
| Triamterene | 15 | 17 | 18 | 21 | 28 | 16 | 19 ± 5 |
| Warfarin | 12 | 12 | 17 | 15 | 20 | 17 | 15 ± 3 |
| Betaine hydrochloride | 6 | 10 | 9 | 17 | 20 | 18 | 13 ± 6 |
| Triflupromazine | 4 | 10 | −2 | 1 | 8 | −6 | 2 ± 6 |
| hydrochloride | | | | | | | |
| Flunixin meglumine | 13 | 15 | 14 | 18 | 19 | 4 | 14 ± 5 |
| Azelastine hydrochloride | 30 | 31 | 33 | 34 | 42 | 30 | 34 ± 4 |
| Toremiphene citrate | 3 | 7 | 13 | 12 | 15 | 11 | 10 ± 4 |
| Pregnenolone succinate | 6 | 12 | 8 | 13 | 17 | 15 | 12 ± 4 |
| Desoxymetasone | 34 | 42 | 34 | 40 | 42 | 41 | 39 ± 4 |
| Buspirone hydrochloride | 0 | −3 | 0 | −1 | −2 | −11 | −3 ± 4 |
| Ethanolamine oleate | 5 | 7 | 8 | 9 | 12 | 0 | 7 ± 4 |
| imethyl fumarate | 3 | −2 | 8 | 7 | 9 | 6 | 5 ± 4 |
| Modaline sulfate | 10 | 7 | 12 | 12 | 13 | 17 | 12 ± 3 |
| Thiostrepton | 14 | 15 | 25 | 27 | 43 | 42 | 28 ± 12 |
| Clomipramine | 0 | 3 | 9 | 3 | 9 | 7 | 5 ± 4 |
| hydrochloride | | | | | | | |
| Tilorone | 4 | 5 | 5 | 11 | 10 | 0 | 6 ± 4 |
| Saxagliptin | 5 | 0 | 6 | 6 | 11 | 3 | 5 ± 4 |
| Gadoteridol | 7 | 3 | 10 | 13 | 16 | 15 | 11 ± 5 |
| Clofarabine | 7 | 8 | 7 | 13 | 20 | 16 | 12 ± 5 |
| Vorinostat | 5 | 13 | 8 | 14 | 18 | 22 | 13 ± 6 |
| Algestone acetophenide | −5 | −2 | 0 | −2 | 3 | −10 | −3 ± 5 |
| Penciclovir | −11 | 1 | 2 | 3 | 7 | −3 | 0 ± 6 |
| Candicidin | 3 | 13 | 17 | 19 | 23 | 18 | 15 ± 7 |
| Protoporphyrin ix | 12 | 16 | 21 | 18 | 28 | 29 | 20 ± 7 |
| Octopamine | 1 | 6 | 12 | 9 | 18 | 17 | 11 ± 6 |
| hydrochloride | | | | | | | |
| Tiratricol | 72 | 71 | 72 | 74 | 77 | 78 | 74 ± 3 |
| Edaravone | 21 | 13 | 13 | 26 | 19 | 6 | 16 ± 7 |
| Sodium tetradecyl sulfate | 9 | 4 | 12 | 12 | 13 | 5 | 9 ± 4 |
| Cetrimonium bromide | 1 | −1 | −4 | 3 | 8 | 2 | 2 ± 4 |
| Miltefosine | 4 | 3 | 11 | 10 | 11 | 11 | 8 ± 4 |

*Plate number.

TABLE 12

| | Total Activity Control | | |
|---|---|---|---|
| Plate # | S/B^{a} | S/N^{b} | Z′^{c} |
| 1 | 2.5 | 55.3 | 0.85 |
| 2 | 2.6 | 38.6 | 0.80 |
| 3 | 2.6 | 40.1 | 0.75 |
| 4 | 2.5 | 99.8 | 0.76 |
| 5 | 2.5 | 107.3 | 0.94 |
| 6 | 2.4 | 56.8 | 0.73 |
| Average | 2.5 | 66 | 0.81 |
| SD^{e} | 0.1 | 30 | 0.08 |

In order to more rigorously identify compounds suitable for further characterization, the threshold for a positive hit was increased to 50% inhibition. The culmination of the screening efforts identified two compounds: benserazide-HCl and 3,3′,5-triiodothyroacetic acid (tiratricol).

Biochemical Characterization of Cif Inhibitors.

The first step in characterizing the two compounds identified by high throughput screening was to verify that the inhibition was reproducible using fresh preparations. Chemical libraries are often stored for extended periods of time, which can lead to breakdown products contributing to the assay outcome. While the inhibition observed with a fresh tiratircol solution was consistent with the primary and secondary screening results, a fresh benserazide-HCl solution lost all observable inhibition. Additionally, it was noticed that benserazide-HCl solutions, either aqueous or in DMSO, acquired a red color over time, suggesting that the compound was susceptible to breakdown.

After aging the freshly prepared benserazide-HCl solution for 3 months at room temperature, Cif inhibition was re-tested. Once more, there was no detectable inhibition. Cocrystallization of Cif protein with the red benserazide hydrochloride breakdown solution resulted in crystals with an enriched color over the well solution, indicating that the chromophore had some affinity for Cif protein. However, clear electron density was not obtainable for any additional compounds bound specifically to Cif. This observation could either be due to low occupancy of a molecule bound to Cif, or a non-specific interaction. 1D proton NMR revealed that a large number of additional peaks were present in the spectra of the breakdown sample. LC/MS analysis detected >100 additional compounds present after aging a benserazide-HCl solution in water. Due to the high diversity of products generated by benserazide-HCl breakdown, as well as the inability to repeat inhibition of Cif EH enzyme activity, this compound was not further investigated.

After successfully recapitulating Cif inhibition with fresh tiratricol, the effect was confirmed with an independent substrate and assay. Using the adrenochrome reporter assay and epoxyhexane, an epoxide substrate previously shown to be hydrolyzed by Cif, a robust inhibition of Cif enzyme activity was observed. Subsequently, tiratricol inhibition was characterized kinetically, once again using the fluorogenic substrate CMNGC. The Kᵢ was found to be 4.1±0.4 μM. Additionally, the Kᵢ was independent of substrate concentration, indicating that tiratricol functioned via a non-competitive mechanism of inhibition.

Structural Studies of Tiratricol Bound to Cif.

To further illuminate the mechanism of inhibition, Cif was crystallized in the presence of tiratricol. A serendipitous benefit of using tiratricol with X-ray diffraction experiments was the presence of three iodine atoms in the compound which could be used to obtain an anomalous signal. Oscillation data for Cif-tiratricol co-crystals were collected at both native and anomalous wavelengths (Table 13).

TABLE 13

| | Wild-Type Cif/Tiratricol | |
|---|---|---|
| Data Collection | | |
| Wavelength (Å) | 1.0000 | 1.7220 |
| Space Group | C2 | C2 |
| Unit Cell Dimensions | | |
| a, b, c (Å) | 169.7, 84.0, 89.5 | 170.0, 84.4, 89.7 |
| α, β, γ (°) | 90, 100.5, 90 | 90, 100.5, 90 |
| Resolution (Å) | 42.23-1.75 | 46.50-2.03 |
| | (1.80-1.75) | (2.08-2.03) |
| R_{sym}^{b} (%) | 8.1 (35.9) | 9.4 (26.2) |
| R_{mrgd-F}^{c} (%) | 10.2 (38.9) | 6.4 (26.2) |
| I/σI | 12.4 (3.7) | 14.2 (4.7) |
| Completeness (%) | 100.0 (100.0) | 98.9 (86.0) |
| Redundancy | 4.2 (4.2) | 7.1 (4.5) |
| SigAno^{d} | | 0.85 (0.71) |
| Refinement | | |
| Total number of reflections | 124409 | |
| Reflections in the test set | 6197 | |
| R_{work}^{e}/R_{free}^{f} (%) | 16.4/19.5 | |
| Number of atoms: | | |
| Protein | 9427 | |
| Solvent | 1168 | |
| Ligand | 84 | |
| Ramachandran plot^{g} (T) | 91.0/8.6/0.4/0 | |
| Bₐᵥ (Å²) | | |
| Protein | 13.6 | |
| Solvent | 28.2 | |
| Bond length RMSD | 0.007 | |
| Bond angle RMSD | 1.105 | |

^{a}Values in parentheses are for data in the highest-resolution shell.
^{b}R_{sym} = ΣₕΣᵢ□I(h) − Iᵢ(h)□/ΣₕΣᵢ Iᵢ(h), where Iᵢ(h) and I(h) values are the i-th and mean measurements of the intensity of reflection h.
^{c}R_{mrgd-F} is robust indicator of the agreement of structure factors of symmetry-related reflections and is described by Diederichs & Karplus (1977) Nat. Struct. Biol. 4(4): 269-75.
^{d}SigAno = <(□F(+) − F(−)□σ_{Δ})>
^{e}R_{work} = Σₕ□F_{obs}(h) − F_{calc}(h)□Σₕ F_{obs}(h), h∈{working set}.
^{f}R_{free} = Σₕ□F_{obs}(h) − F_{calc}(h)□Σₕ F_{obs}(h), h∈{test set}.
^{g}Core/allowed/generously allowed/disallowed.

An isomorphous difference map calculated against the apo-protein also revealed the prominent peaks per Cif molecule. An anomalous electron density map also exhibited three strong peaks per Cif molecule at the same positions observed in the difference map. Prior to inclusion in the model, a 2F_{O}−F_{C} map displayed unambiguous electron density for tiratricol bound within the active site tunnel of Cif, and the iodine positions were consistent with the anomalous and isomorphous difference peaks. Tiratricol binding occludes the tunnel by which a substrate enters the active site of Cif, preventing substrates from reaching the catalytic machinery. As tiratricol and an epoxide substrate bind at distinct sites within the protein, this finding is consistent with the kinetic observation of a non-competitive inhibition.

Furthermore, the tiratricol bound structure of. Cif revealed the mechanism by which access to the active site is gated. In the apo structure of Cif (PDB ID 3 KD2), no direct pathway to the active site was entirely present. The location of this tunnel based on the most accessible route to the protein surface has been predicted. However, this route is constricted to be <0.5 Å wide at the narrowest point. This opening is smaller than the van der Waals radius of carbon, indicating that it is not broad enough for a substrate to traverse in this conformation. Tiratricol binding captured the open conformation of this tunnel. Gating appeared to be controlled by three residues: Phe164, Leu174, and Met272. These residues form a gasket that can restrict access to the active site, and may also act as a selectivity filter for epoxide substrates. Through their motion, the tunnel opens to create a ≧3.4 Å wide passageway. It was interesting to note that there only appeared to be two prominent interactions between Cif and tiratricol. The first was a hydrogen bond to the backbone carbonyl of Gly270. The second was with gating residue Phe164, which had rotated the phenyl group of its side chain to participate in a n-stacking interaction with an aromatic ring of tiratricol.

The Effect of Tiratricol on Cif Expression by P. aeruginosa.

Cif is expressed by P. aeruginosa as part of a three gene operon. This operon is negatively regulated by CifR, an epoxide sensitive TetR family repressor (MacEachran, et al. (2008) supra). CifR binds to DNA and prevents the transcriptional machinery from accessing the promoter for the Cif operon. Upon exposure to an epoxide containing molecule, CifR releases DNA, resulting in increased transcription of the Cif operon and increased Cif protein production. It has been previously shown that Cif and CifR have overlapping substrate/ligand specificities (MacEachran, et al. (2008) supra). Therefore, it is important to consider any potential Cif inhibitor in the context of the bacterial response. If an inhibitor were to trigger P. aeruginosa to produce more Cif, the inhibitor concentrations could potentially be overwhelmed by excess Cif protein. To investigate this possibility, tiratricol was tested for the ability to bind CifR and promote its release from DNA by means of an in vitro DNA binding assay. Tiratricol caused CifR to release from DNA as efficiently as a previously characterized epoxide ligand. This was not unexpected considering that many TetR family repressors have broad ligand binding specificities as well as multiple ligand binding sites (Schumacher & Brennan (2003) Res. Microbiol. 154:69-77). An important factor in determining if an exogenous compound will induce gene expression is its accessibility to the bacterial cytosol. Therefore, Cif protein levels were examined after P. aeruginosa was exposed to tiratricol. In contrast to the in vitro DNA binding assay result, there was no detectable increase in Cif expression using this in vivo model.

### Example 3

### Structure Activity Relationship Analysis of Tiratricol

Table 14 lists the activity of various tiratricol analogs for inhibiting Cif.

TABLE 14

| | | | CIF IC₅₀ |
|---|---|---|---|
| Entry | Structure | MW g/mol | (μM)^{a} |
| Tiratricol | 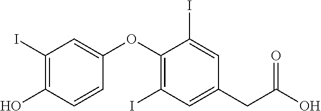 [SMILES: O=C(O)Cc1cc(I)c(Oc2ccc(O)c(I)c2)c(I)c1] | 621.8 | 4.7 ± 0.6 |
| 1 | 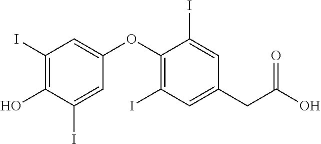 [SMILES: O=C(O)Cc1cc(I)c(Oc2cc(I)c(O)c(I)c2)c(I)c1] | 747.8 | 20 ± 2 |
| 2 | 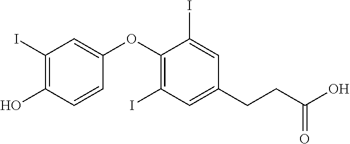 [SMILES: O=C(O)CCc1cc(I)c(Oc2ccc(O)c(I)c2)c(I)c1] | 635.8 | 4.4 ± 0.5 |
| 3 | 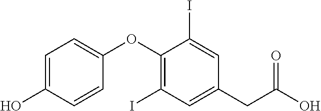 [SMILES: O=C(O)Cc1cc(I)c(Oc2ccc(O)cc2)c(I)c1] | 496.0 | 100 ± 2 |
| 4 | 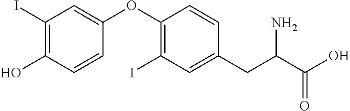 [SMILES: NC(Cc1ccc(Oc2ccc(O)c(I)c2)c(I)c1)C(=O)O] | 525.1 | 80 ± 10 |
| 5 | 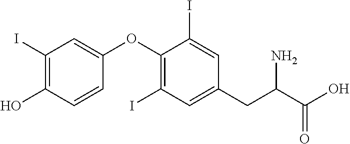 [SMILES: NC(Cc1cc(I)c(Oc2ccc(O)c(I)c2)c(I)c1)C(=O)O] | 651.0 | 21 ± 2 |
| 6 | 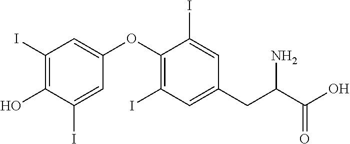 [SMILES: NC(Cc1cc(I)c(Oc2cc(I)c(O)c(I)c2)c(I)c1)C(=O)O] | 776.9 | >100 |
| 7 | 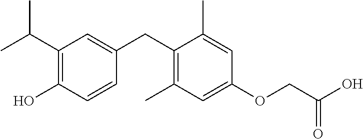 [SMILES: Cc1cc(OCC(=O)O)cc(C)c1Cc1ccc(O)c(C(C)C)c1] | 328.4 | >100 |
| 8^{b} | 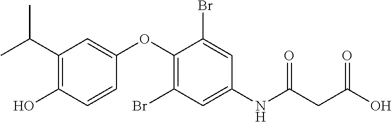 [SMILES: CC(C)c1cc(Oc2c(Br)cc(NC(=O)CC(=O)O)cc2Br)ccc1O] | 487.1 | 2.7 ± 0.4 |
| 9 | 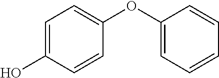 [SMILES: Oc1ccc(Oc2ccccc2)cc1] | 186.2 | >100 |
| 10 | 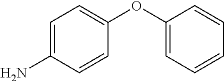 [SMILES: Nc1ccc(Oc2ccccc2)cc1] | 185.2 | >100 |
| 11 | 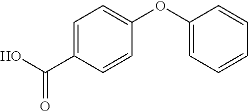 [SMILES: O=C(O)c1ccc(Oc2ccccc2)cc1] | 214.2 | >100 |

Conditions: [E] = 0.6 μM; [S] = 25 μM; 37° C. for 15 minutes; pre-incubation 5 minutes at 37° C.
^{a}average ± standard deviation (n = 3).
^{b}also known as KB2115 (Karo Bio).

This analysis indicated that the addition or removal of an iodine to tiratricol (entries 2 and 4) reduced potency. While having a longer acid arm (entry 3) did not change the potency, addition of an amine (entries 5 and 6) reduced potency. Simple molecules without side chains (entries 9 to 11) did not exhibit any inhibition. In addition, thyroid hormone mimetics (entries 7 and 8) were tested and while the activity of entry 7 was not good, entry 8 was very good.

### Example 4

### Additional Cif Inhibitors

To identify additional inhibitors of the Cif enzyme, further screening assays were conducted. The results of these screens identified two additional classes of Cif inhibitors: urea-based scaffolds and endogenous epoxide scaffolds.

Urea-Based Scaffolds.

A screen of soluble expoxide hydrolase (sHE) inhibitors identified the urea-based scaffold shown in Table 15.

TABLE 15

| | Inhibition | CIF | HsEH | |
|---|---|---|---|---|
| | at 10 μM | IC₅₀ | IC₅₀ | |
| Entry | (%) | (μM) | (nM) | Structure |
| 1 | 41 | >100 | 3.8 | 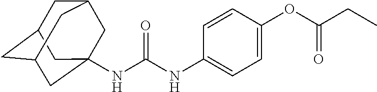 [SMILES: CCC(=O)Oc1ccc(NC(=O)NC23CC4CC(CC(C4)C2)C3)cc1] |
| 2 | 41 | NA | | 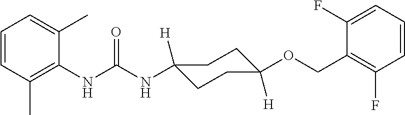 [SMILES: Cc1cccc(C)c1NC(=O)NC1CCC(OCc2c(F)cccc2F)CC1] |
| 3 | 39 | 1.8 ± 0.4 | | 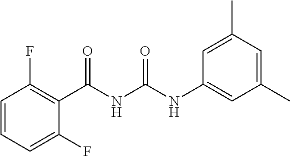 [SMILES: Cc1cc(C)cc(NC(=O)NC(=O)c2c(F)cccc2F)c1] |
| 4 | 50 | 1.5 ± 0.3 | | 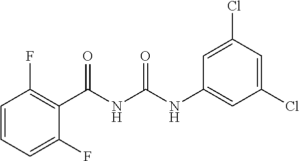 [SMILES: O=C(NC(=O)c1c(F)cccc1F)Nc1cc(Cl)cc(Cl)c1] |
| 5 | 39 | >100 | | 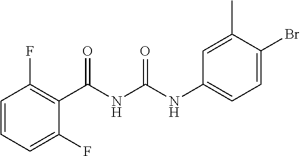 [SMILES: Cc1cc(NC(=O)NC(=O)c2c(F)cccc2F)ccc1Br] |
| 6 | 38 | >100 | | 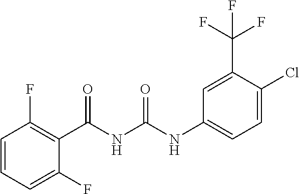 [SMILES: O=C(NC(=O)c1c(F)cccc1F)Nc1ccc(Cl)c(C(F)(F)F)c1] |
| 7 | 40 | >100 | | 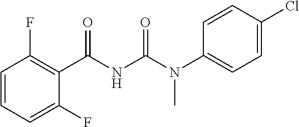 [SMILES: CN(C(=O)NC(=O)c1c(F)cccc1F)c1ccc(Cl)cc1] |
| 8 | 44 | N.A. | 33300 | 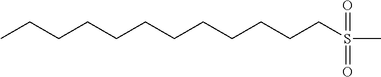 [SMILES: CCCCCCCCCCCCS(C)(=O)=O] |
| 9 | 38 | 7.4 ± 0.6 | 100000 | 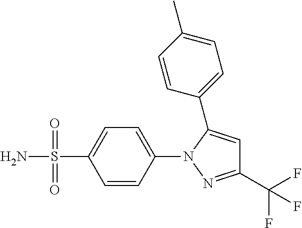 [SMILES: Cc1ccc(-c2cc(C(F)(F)F)nn2-c2ccc(S(N)(=O)=O)cc2)cc1] |
| 10 | 44 | 40 ± 6 | 38 | 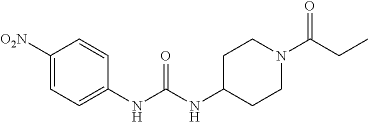 [SMILES: CCC(=O)N1CCC(NC(=O)Nc2ccc([N+](=O)[O-])cc2)CC1] |

The results of this analysis indicated that entries and 4 exhibited IC₅₀ values even more potent than tiratricol.

Endogenous Epoxide Scaffolds.

Detailed kinetic information on the interaction of Cif with an endogenous arachidonic acid-derived signaling epoxide, 14,15-EET (14,15-epoxyeicosa-5,8,11-trienoic acid), was also obtained.

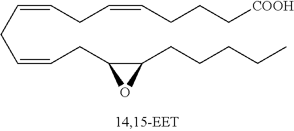 [SMILES: CCCCC[C@H]1O[C@H]1C/C=C\C/C=C\C/C=C\CCCC(=O)O]

Specifically, three concentrations of 14,15-EET were used (0.5, 5 and 50 μM), along with two different incubation times (90 and 180 minutes) and a low enzyme concentration ([E]=0.2 μM). The results were similar at 90 and 180 minutes, confirming that the assay was within the linear range of catalysis. Less than 5% of the substrate was converted: [S]=0.5 μM, v=0.003 pmol/minute; [S]=5 μM, v=0.02 pmol/minute; and [S]=50 μM, v=1.2 pmol/minute.

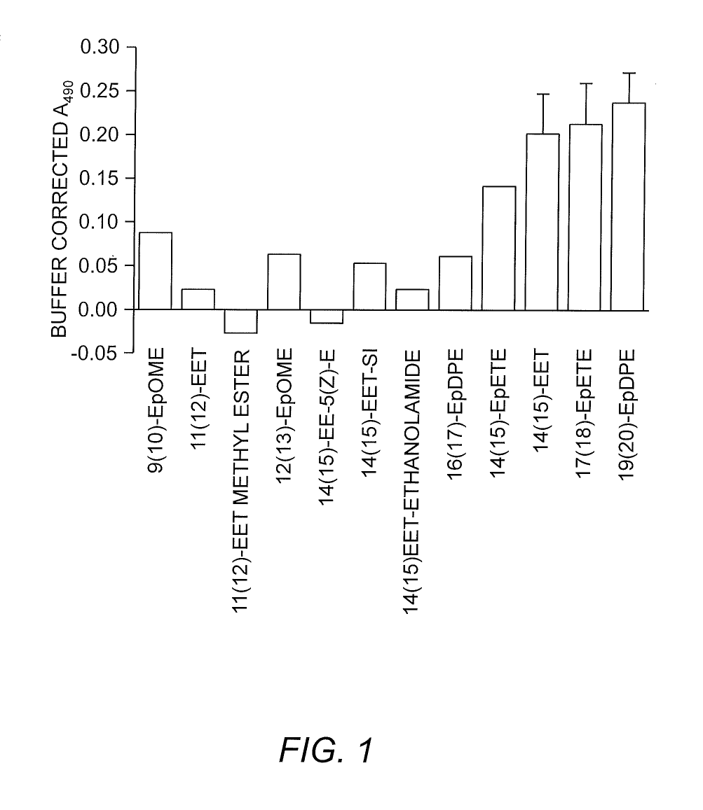

Similar analysis was carried out with various other monoepoxide fatty acids and derivatives therefore, including 9(10)-EpOME, 11(12)-EET, 11(12)-EET methyl ester, 12(13)-EpOME, 14(15)-EE-5(Z)-E, 14(15)-EET-sulfonimide (SI), 14(15)-EET ethanolamide, 16(17)-EpDPE, 14(15)-EpETE, 14(15)-EET, 17(18)-EpETE, and 19(20)-EpDPE. The results of this analysis are presented in FIG. 1.

1. A pharmaceutical composition formulated for pulmonary administration comprising an inhibitor of Cystic fibrosis transmembrane conductance regulator Inhibitory Factor (Cif) activity in admixture with a pharmaceutically acceptable carrier, wherein the inhibitor is a long chain or very long chain fatty acid monoepoxide or has the structure of Formula I 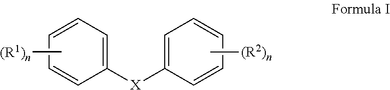
wherein
X is absent or present and when present is —O—, —NH—, —S—, —CH₂—, —NHC(O)NH—, or —C(O)NHC(O)NH—;
n is 0 to 5; and
R¹ and R² are substituted one or more times anywhere on their respective rings, wherein each occurrence of R¹ and R² is independently a hydrogen, hydroxyl, amino, cyano, halo, nitro, mercapto, phosphate, —CH(CH₃)₂—COOH, —CO₂Me, CONH₂, —CONHNH₂, —NHC(O)CH₂COOH, —OCH₂COOH, —OC(O)CH₂CH₃, alkyl, alkenyl, alkynyl, aryl, or amido group.

2-4. (canceled)

5. A method for ameliorating or treating a respiratory disease, or a secondary infection thereof, comprising administering to a subject in need of treatment the pharmaceutical composition of claim 1, thereby ameliorating or treating the subject's respiratory disease, or secondary infection thereof.

6-7. (canceled)

8. A method for identifying an inhibitor of Cystic fibrosis transmembrane conductance regulator Inhibitory Factor (Cif) activity comprising
contacting a prokaryotic Cif protein with a test compound in the presence of cyano(6-methoxynaphthalen-2-yl)methyl (oxiran-2-ylmethyl) (CMNGC); and
determining whether the test compound inhibits hydrolysis of CMNGC, thereby identifying an inhibitor of Cif activity.

9. An inhibitor identified by the method of claim 8.

10. A method for inhibiting the activity of Cystic fibrosis transmembrane conductance regulator Inhibitory Factor (Cif) activity comprising contacting Cif with a long chain or very long chain fatty acid monoepoxide or an inhibitor having the structure of Formula I, 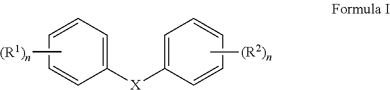
wherein
X is absent or present and when present is —O—, —NH—, —S—, —CH₂—, —NHC(O)NH—, or —C(O)NHC(O)NH—;
n is 0 to 5; and
R¹ and R² are substituted one or more times anywhere on their respective rings, wherein each occurrence of R¹ and R² is independently a hydrogen, hydroxyl, amino, cyano, halo, nitro, mercapto, phosphate, —CH(CH₃)₂—COOH, —CO₂Me, CONH₂, —CONHNH₂, —NHC(O)CH₂COOH, —OCH₂COOH, —OC(O)CH₂CH₃, alkyl, alkenyl, alkynyl, aryl, or amido group.

11-13. (canceled)
